(19) United States
(12) Patent Application Publication
(10) Pub. No.: US 2009/0137063 A1
(43) Pub. Date: May 28, 2009

(54) METHOD FOR DETERMINING THE QUALITY OF A BIOLOGICAL SAMPLE

(76) Inventors: Karl Skold, Uppsala (SE); Marcus Svensson, Uppsala (SE); Per Andren, Uppsala (SE); Per Svenningsson, Stockholm (SE)

Correspondence Address: MANELLI DENISON & SELTER, 2000 M STREET NW SUITE 700, WASHINGTON, DC, 20036-3307, US

(21) Appl. No.: 12/091,344

(22) PCT Filed: Nov. 28, 2006
(86) PCT No.: PCT/SE2006/050518

§ 371 Date: May 30, 2008

Related U.S. Application Data
(60) Provisional application No. 60/740,542, filed on Nov. 29, 2005.

(30) Foreign Application Priority Data
Nov. 29, 2005 (SE) 0502648-9

(51) Int. Cl. G01N 33/566; G01N 33/68; G01N 27/26; B01D 59/44

(52) U.S. Cl. 436/501; 436/34; 250/282; 204/461

(57) ABSTRACT

The present invention relates to a method of determining the quality of a biological sample. A method according to the invention may be used to determine if a biological sample is suitable to use in a further biological assay demanding samples of good quality to render an accurate result. The method comprises detecting the presence of protein fragments in the biological sample by using appropriate means. The invention also relates to a kit for determining the quality of a biological sample.

### FIELD OF THE INVENTION

The present invention relates to the field of biological analyses, and more specifically to a method for determining the quality of a biological sample. A method according to the invention may be used to determine if a biological sample is suitable, i.e. of good enough quality, to be used in a further biological analysis, such as in a pharmacological analysis, which often is costly to perform.

### INTRODUCTION

Proteins and peptides in tissues, cells, or extracellular fluids such as blood, plasma, and urine are widely investigated by methods involving electrophoresis, chromatography, and mass spectrometry (MS). The concentration of the individual proteins and peptides in most unrefined samples spans at least 10 orders of magnitude, which limits the simultaneous protein detection, e.g., by using two-dimensional gel electrophoresis (2D-GE). The low abundant proteins, hormones, and neuropeptides are overwhelmed by the abundance of a few very high abundant proteins. Other techniques, such as two-dimensional liquid chromatography (2D-LC) coupled MS, focused on the small protein content of biological samples, enable the identification and characterisation of low abundant polypeptides. This puts great demands on sample handling and sample quality and will expose the fast degradation of some proteins.

Post-sampling activity of proteases has been shown to play an important role on the peptide levels of the brain, as well as for detecting post-translational modifications of proteins and peptides (Sköld et al. 2002, Svensson et al 2003). The peptide and protein content in brain tissue is greatly influenced by the sample handling methods and by the time-interval from death or sampling to the inactivation of proteolytic enzymes. Previous comparisons of post mortem tissue or body fluids have aimed on longer time spans and/or are often focused on the temperature in which the samples are stored (Fountoulakis et al. 2001, Sabudeco et al. 2003, Khosravi et al. 2005, Franzen et al. 2003).

Franzen et al. (2003) studied post mortem effects on proteins using 2D-GE and mass spetrometric methods. This study suggested that the degradation of dihydropyrimidinase related protein-2 protein could be a good marker of post mortem time and temperature. However, the degradation of the proteins was studied within hours after sampling and not within minutes, leaving questions about changes in protein degradation within minutes unanswered. Furthermore, Fountoulakis et al. studied protein level alterations in rat brains using 2D-GE and mass spectrometry. Also in this study, alterations in protein levels where studied several hours post mortem, not detecting the changes in protein levels the very first minutes after obtaining the sample from its source.

US 2002/0197741 discloses a method for determining the time of death using the degradation of Cardiac Troponin (cTn1) as a specific marker. Standard curves of degradation of the protein tropomin1 were used to predict time of death. It is not suggested or implied that cTn1 could be used in a method for determining the quality of a sample.

Che et al. (2005), describes a method wherein protein degradation was prevented in situ in the brain utilizing a standard microwave oven after mice had been sacrificed by decapitation. This study detected some protein degradation fragments after the microwave treatment. The authors stated that these fragments appear to result from protein breakdown caused by the sample preparation and not from an enzymatic reaction during the post-mortem period. However, it was not possible to verify this statement, since a comparison using focused microwave irradiation in vivo with the proposed method was not performed.

Khosravi et al (2005) studied insulin-like growth factor (IGF-I) using different assay methodologies in various fresh and stored serum samples. In this study the stability of IGF-I was analyzed by immunological methods, such as the enzyme linked immunosorbent assay (ELISA), radioimmunoassay (RIA) and immunoradiometric assay (IRMA). It was found that IGF-I levels in fresh serum samples, which were stored in −4° C. up to 48 hrs before freezing and assayed within a week, by all methods were similar and highly correlated. In contrast, in the old frozen samples, which were stored 2-8 years and subsequently went through two freeze and thaw cycles within 1 week, the inter-method median IGF-I levels were decreased and varied 3- to 4-fold and the values were poorly correlated. It was concluded by the authors that the low IGF-I levels may be indicative of questionable sample quality. Obviously, this study was not studying the short term effects of serum sample handling.

WO 2006/005622 A1 discloses methods for determining and monitoring sample quality comprising the addition of a standard sensitive to degradation.

The above described attempts leave an unfulfilled long felt need for providing a way to specifically determine the quality of a biological sample, which is to be used in a further biological assay. The known methods of analyzing protein degradation products are not focused on the importance of the time aspect until inactivation of the sample after obtaining it from its in vivo natural environment or a place where it is kept in an inactivated state. Using a sample of good quality in an assay provides a cost-effective and money-saving alternative for many users, e.g., pharmaceutical companies.

### SUMMARY OF THE INVENTION

The present invention relates to a method of determining the quality of a biological sample which has been made biologically inactive, wherein said quality is determined by measuring the degradation of one or more proteins and/or peptides present in said sample by detecting the presence of a degradation product of said protein and/or peptide in said sample after inactivation. To determine the quality of the sample, the detected amount of degradation products may be compared to the amount of intact proteins in said sample. Another object of the present invention relates to determining the quality of a biological sample by measuring the degradation of the protein stathmin, which by the present inventors has been identified as one of the first proteins to be degraded post sampling in vitro.

Furthermore, the present invention relates to a kit comprising means for detecting the presence of protein fragments, such as peptide fragments of stathmin or acetylated N-terminal fragments of proteins or protein fragments that have been modified in another way, in a biological sample. Said kit may comprise a site recognition molecule, such as an antibody, for detecting the presence of a protein and/or a peptide or a fragment thereof in a biological sample.

The present inventors show that the quality of a biological sample is determined by the initial handling procedure of the sample i.e., the handling of the sample when it has been taken from its in vivo source or from a place where it is kept in a temporarily inactive state e.g., in a freezer, until the sample has been proteolytically deactivated, as the degradation of naturally occurring proteins start immediately after the sample has been removed from its in vivo source or a place where it is kept in a temporarily inactive state. An inactivation of the sample needs to be performed in close proximity to removal of the sample from its source. Such an inactivation may be performed by a variety of means, such as by heating, mechanical treatment or by chemicals.

The present invention provides means for determining the quality of a biological sample, which has been proteolytically inactivated. This invention is useful for deciding if a sample is suitable for use in further biological analyses, such as in pharmacological or biochemical analyses, when it is important that the sample is of high quality to produce accurate results.

### BRIEF DESCRIPTION OF THE DRAWINGS

FIG. 1. The ion intensity of the 19-amino acid residue peptide fragment from the N-terminal end of stathmin with a molecular weight of 2105 Da, (SEQ ID NO:2) detected in striatum of mice, 0, 1, 3, 10 min post sampling.

FIG. 2. Panel A-B show segments of the ESI MS elution profile of peptides from mouse hypothalamus at 0 and 10 min, respectively (18.5-30.5 minutes in the m/z range 455-726). Some of the identified neuropeptides are indicated in panel A. Spot intensity is represented by color change, where black has the highest intensity and white the lowest. Panel C-D is the same area as A-B shown in three dimensions.

FIG. 3. Panel A shows the unphosphorylated form of corticotrophin-like intermediate lobe peptide (CLIP) that decrease slowly post mortem. Panel B shows the relative levels of phosphorylated CLIP that decrease more rapidly post mortem. The post mortem times are 0, 1, 3, 10 minutes.

FIG. 4. The experimental setup of the nano LCMS experiment.

FIG. 5. The 19-amino acid residue peptide fragment from the N-terminal end of stathmin with a molecular weight of 2105 Da (SEQ ID NO:2) detected in plasma using nano LCMS, visualized in two dimensions, where spot intensity is represented by color change, black being the most intense reading and white the lowest.

FIG. 6. The relative intensity of three different peptides from hypothalamic tissue (mouse), which decrease over time, 0, 1, 3, 10 minutes post sampling. Panel A shows the ion intensity of leucine enkephalin, panel B shows the ion intensity of a peptide from pro-opiomelanocortin (POMC), and panel C shows the ion intensity of beta-endorphin.

FIG. 7. Image of a two dimensional gel electrophoresis (2D-GE) separation of proteins from mouse cortex (focused microwaved tissue).

FIG. 8. Total ion chromatograms from a nanoLC MS analysis of mouse striatum shown in the m/z range of 300-1000 over a 60 min gradient elution. The y-axis indicates relative intensity, 0-100%, and the x-axis shows elution time (min). The post mortem times are, A) 0 min, B) 1 min, C) 3 min, and D) 10 min. By using pattern recognition it is possible to decide the quality of the sample by examining the total ion chromatograms.

### DEFINITIONS

In one context of the present invention, the term “post sampling” time refers to the period of time after a biological material has been separated from its natural in vivo environment, and/or after the biological material has been removed from storage where it is kept in a temporarily inactive state i.e., where it is not subject to degradation, such as, but not limited to, a freezer. Post sampling, the biological material i.e., the biological sample, is sensitive to degradation, such as proteolytic degradation, unless an inactivation of the sample takes place. The term “post sampling” may also in one context of the present invention relate to the time which has passed after the sample has been taken from its natural in vivo source, and until the sample has been placed for storage in a temporarily inactive state, such as in liquid nitrogen. When the sample is removed from storage in a temporarily inactive state, it is appropriate if the sample is inactivated immediately, such as within a few seconds, so that the post sampling time is approximately only the time which has passed from when the biological sample was taken from its natural in vivo source, until when it was placed, e.g., in liquid nitrogen, where it was kept in a temporarily inactive state. In another context of the invention, these two time periods are added to give the “post sampling” time.

When the term “quality” is referred to in the context of a “biological sample” according to the invention, it is in one context referring to a biological status of the sample, i.e., to what extent degradation of the naturally occurring proteins in the sample has occurred. The quality of a sample may be determined by comparing the amount of fragmented protein with the amount of intact protein, i.e., by analyzing the ratio between fragmented protein and intact protein, in the sample. Such a ratio may vary between different tissues and different proteins and protein fragments thereof, which are studied in a method according to the invention. When specific proteins are used as markers for degradation, protein specific molecules, such as antibodies, may in one context of the invention be used to detect both intact protein and/or protein fragments. When the intact protein is detected but essentially no fragments can be detected the sample has been handled properly and is of high quality. When fragments are detected a ratio may be calculated e.g., between fragments of stathmin and intact stathmin. The ratio may be compared with a “temporal degradation profile” defined for different tissues and/or body fluids. The ratio may be compared to a standard for proteins present in different in vivo tissues and/or bodily fluids. In one embodiment of the invention, a biological sample is considered to be of a high quality when the ratio between the amount of one or more specific degraded proteins and the amount of the corresponding intact proteins in said sample do not substantially exceed the ratio from a sample that is collected from the same kind of tissue or body fluid that has immediately been inactivated post sampling. Deactivated samples are protected from further proteolytical activity of proteases, but may still be exposed to other physical aspects, such as oxidation of methionine residues.

In another context of the invention, the “quality” of a biological sample may be determined by its ability to resemble a native/in vivo condition of a tissue or body fluid, and/or its suitability for representing in vivo tissue or in vivo body fluid.

A “biological sample” according to the invention, means a sample which is of biological origin. A biological sample for use in the present invention may originate from any biological organism, such as, but not limited to, a vertebrate, (e.g., a mammal or a human), an invertebrate, a plant or a microorganism. A biological sample originates from any part, i.e., tissue and/or bodily fluid, of said biological organism, such as, but not limited to, epithelial tissue, connective tissue, muscle tissue and/or nervous tissue, e.g., lung, skin, heart, bone, intestine, breast, uterus, ovaries, brain, endometrium, cervix, colon, esophagus, stomach, hepatocellular, kidney, spleen, mouth, prostate, liver, testicles, endocrine tissue, thyroid, blood, plasma, serum, lymph, saliva, urine, feces, ascites, tears, saliva, and/or brain cerebrospinal fluid.

“Stathmin” may in the context of the present invention, refer to SEQ ID NO:1 (human), SEQ ID NO:3 (mouse) and/or SEQ ID NO:4 (rat).

In the present context “degradation”, the term refers to the process wherein a protein present in a biological sample is digested or divided into smaller fragments e.g., due to the presence of enzymes, such as proteases, in the sample. Degradation may also be due to changes in temperature, pH or humidity in the environment where the sample is kept. The presence of degraded proteins in a biological sample is seen as an indication of deteriorating quality of the sample. Degradation may also refer to modifications occurring post sampling, such as oxidation or the loss of phosphorylations, glycosylations or other post-translational modifications.

A “protein” is a biological macromolecule constituted by amino acid residues linked together by peptide bonds. Proteins, as linear polymers of amino acids, are also called polypeptides. Typically, proteins have 50-800 amino acid residues and hence have molecular weights in the range of from about 6,000 to about several hundred thousand Dalton or more. Small proteins are called peptides or oligopeptides.

A “neuropeptide” is a member of a class of protein-like molecules present in the brain. Neuropeptides often consist of short chains of amino acids, with some functioning as neurotransmitters and some functioning as hormones. Examples of neuropeptides are endorphins and enkephalins.

An “acetylated protein” refers to a protein that is acetylated in its N-terminus. N-terminal acetylation occurs post-translationally on eukaryotic cytoplasmic proteins to protect them from N-terminal degradation.

An “inactivation” of a biological sample according to the invention, refers to a procedure wherein said sample is inactivated, i.e., the degradation of the proteins present in the sample is stopped by means of various treatments of the sample such as, but not limited to, heating, mechanical and chemical treatment, etc., as well as by other means disclosed by the present invention and/or known to the skilled person. During such a process, some proteins are denaturated, including proteases which are involved in the degradation process of the proteins present in the sample. In one aspect, a method according to the invention comprises inactivating a biological sample prior to determining the quality of the sample.

A “biologically inactivated” sample according to the invention, is referring to a sample wherein the degradation of the proteins in the sample has been stopped by a process such as, but not limited to, heating, mechanical treatment, chemical means, etc.

A “site recognition molecule” according to the present invention, means a molecule, such as, but not limited to, an antibody that recognizes a specific binding site unique to its target.

“Denaturation” is commonly defined as any noncovalent change in the structure of a protein. This change may alter the secondary, tertiary or quaternary structure of the molecules. Since denaturation reactions are not strong enough to break the peptide bonds, the primary structure (sequence of amino acids) remains the same after a denaturation process. Denaturation disrupts the normal alpha-helices and beta sheets in a protein and uncoils it into a random shape. When using this definition it should be noted that what constitutes denaturation is largely dependent upon the method utilized to observe the protein molecule. Some methods can detect very slight changes in structure while others require rather large alterations in structure before changes are observed. For those proteins that are enzymes, denaturation can be defined as the loss of enough structure to render the enzyme inactive. Changes in the rate of the reaction, the affinity for substrate, pH optimum, temperature optimum, specificity of reaction, etc., may be affected by denaturation of enzyme molecules.

A “temporal degradation profile” refers to a characteristic profile of a protein in a sample originating from a tissue or a bodily fluid. The protein is degraded in the tissue or bodily fluid post sampling producing degradation products which are characteristic of that specific protein in a specific tissue and/or body fluid. A temporal degradation profile may in the context of the present invention be used to determine if a sample is of good quality. An example of a protein with a temporal degradation profile is stathmin described in SEQ ID NO:1, SEQ ID NO: 3, and SEQ ID NO:4.

SEQUENCES
SEQ ID NO:1: Human stathmin
(UniProtKB/Swiss-Prot entry P16949 [STMN1_HUMAN]
Stathmin ExPASy Home)
ASSDIQVKELEKRASGQAFELILSPRSKESVPEFPLSPPKKKDLSLEEIQ
KKLEAAEERRKSHEAEVLKQLAEKREHEKEVLQKAIEENNNFSKMAEEKL
THKMEANKEN REAQMAAKLERLREKDKHIE EVRKNKESKDPADETEAD
SEQ ID NO:2 Fragment of stathmin
Ac-ASSDIQVKELEKRASGQAF
SEQ ID NO:3: Mouse stathmin
(UniProtKB/Swiss-Prot entry P54227 [STMN1_MOUSE])
ASSDIQVKELEKRASGQAFELILSPRSKESVPDFPLSPPKKKDLSLEEIQ
KKLEAAEERRKSHEAEVLKQLAEKREHEKEVLQKAIEENNNFSKMAEEKL
THKMEANKEN REAQMAAKLERLREKDKHVE EVRKNKESKDPADETEAD
SEQ ID NO:4: Rat stathmin
(UniProtKB/Swiss-Prot entry P13668 [STMN1_RAT]
Stathmin ExPASy Home)
ASSDIQVKELEKRASGQAFELILSPRSKESVPEFPLSPPKKKDLSLEEIQ
KKLEAAEERRKSHEAEVLKQLAEKREHEKEVLQKAIEENNNFSKMAEEKL
THKMEANKEN REAQMAAKLERLREKDKHVE EVRKNKESKDPADETEAD

### DETAILED DESCRIPTION OF THE INVENTION

The inventors have surprisingly shown that degradation of peptides and proteins in a biological sample occurs almost immediately post sampling, by using the analytical techniques nanoLC coupled to MS. In the sample preparation protocol the proteases were deactivated by thermal energy immediately (in vivo) within 1.4 s, and at 1, 3, and 10 min post mortem. The sample was fractionated using a spin filter, separating molecules per size (Sköld et al. 2002). Molecules less than 10000 Da, (peptides, small proteins and protein fragments) were analyzed and the ratio of intact proteins and protein fragments was compared. The analysis revealed an increasing number in a reproducible manner of protein fragments over time. A peptide fragment from stathmin, a phosphoprotein that can be found in all tissues and most body fluids, is now shown to be an indicator of sample quality, i.e., the fragment displays a stable increase over post sampling time. An over-representation of fragments with their N-terminal blocked by acetylation was also observed. Based on this knowledge, it was possible to use different analytical methods to determine the degradation in a sample, and thereby the sample quality.

Accordingly, the present invention relates to a method for identifying a biological marker for the quality of a biological sample comprising the steps;
detecting the presence and amount of degradation products of proteins and peptides in a test sample at one or more time points within 10 minutes post sampling, such as at 1 minute, 3 minutes and 10 minutes post sampling,
   and
identifying a degradation product which is formed in a time dependent manner within 10 minutes post sampling, such as within 1 minute or within 3 minutes post sampling, as a biological marker for the quality of a biological sample.

In one embodiment, the invention relates to a method for identifying a biological marker for the quality of a biological sample wherein said detection is performed using mass spectrometry. In one further embodiment, said detection is performed by gel electrophoresis alone, or in combination with mass spectrometry. In one preferred embodiment said detection is performed by two-dimensional difference gel electrophoresis (2D DIGE) alone, or in combination with matrix-assisted laser desorption ionization mass spectrometry. In another preferred embodiment, said detection is performed by liquid chromatography alone, or in combination with mass spectrometry. In yet another preferred embodiment, said detection is performed by capillary nanoscale liquid chromatography alone or in combination with electrospray ionization (quadrupole) time-of-flight (nanoLC/ESI Q-TOF) MS. In another preferred embodiment, said detection is performed using immunological methods using antibodies directed to one or more of said protein, peptide and/or degradation product.

In one embodiment, the invention relates to a method for identifying a biological marker for the quality of a biological sample wherein said sample originates from a tissue or a bodily fluid.

For certain types of biological samples where separation procedures are needed to obtain the sample, such as plasma and serum samples, it may be necessary to detect and identify degradation products that are formed during longer time intervals post sampling in order to identify suitable biological markers for the quality of such samples.

Hence, in one embodiment, the invention relates to a method for identifying a biological marker for the quality of a biological sample comprising the steps;
detecting the presence and amount of degradation products of proteins and peptides in a test sample at one or more time points within 60 minutes post sampling, such as at 10 minutes, 20 minutes, 30 minutes, 45 minutes, and 60 minutes post sampling,
   and
identifying a degradation product which is formed in a time dependent manner within 60 minutes post sampling, such as within 10 minutes, 20 minutes, 30 minutes, 45 minutes post sampling, as a biological marker for the quality of a biological sample.

Another object of the present invention is to provide a biological marker for determining the quality of a biological sample, said biological marker characterized by;
being formed post sampling as a degradation product of a protein or a peptide present in said biological sample,
   and
being formed in a time dependent manner within 10 minutes post sampling, such as within 1 minute or within 3 minutes post sampling, in a comparable untreated test sample at 25° C.

In one embodiment of the invention, said biological marker is formed as a degradation product of a protein or peptide having a distinctive temporal degradation profile. In another embodiment said peptide is a neuropeptide. In yet another embodiment said protein is an acetylated protein.

In another embodiment of the invention, the biological marker is a peptide fragment, preferably a fragment of the protein stathmin, and even more preferably an N-terminal fragment of the protein stathmin, and even more preferably said biological marker is the peptide SEQ ID NO: 2.

In another embodiment of the invention, the biological marker is a peptide selected from SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, and SEQ ID NO: 85.

For certain types of biological samples where separation procedures are needed to obtain the sample, such as plasma and serum samples, it may be necessary to use biological markers that are formed during longer time intervals post sampling.

Hence, in one embodiment, the present invention provides a biological marker for determining the quality of a biological sample, said biological marker characterized by;
being formed post sampling as a degradation product of a protein or a peptide present in said biological sample,
   and
being formed in a time dependent manner within 60 minutes post sampling, such as within 10 minutes, 20 minutes, 30 minutes, or 45 minutes, post sampling, in a comparable untreated test sample at 25° C.

Another object of the present invention relates to a method for determining the quality of a biological sample which has been made biologically inactive, wherein said quality is determined by measuring the degradation of one or more proteins and/or peptides present in said sample by detecting the presence of a fragment of said protein and/or peptide in the sample after inactivation. In one embodiment, said quality is determined by comparing the amount of fragmented protein with the amount of intact protein in said sample. One may optionally choose to study one or several proteins and/or fragments thereof simultaneously, in a method according to the invention. In one embodiment, a biological sample originates from a tissue and/or a bodily fluid. Said sample may be inactivated by any appropriate means, such as, but not limited to, heating, mechanical treatment and/or by chemical means.

Another object of the present invention relates to a method for determining the quality of a biological sample, wherein said quality is determined by detecting the presence and/or amount of a degradation product of a protein or a peptide, where said degradation product has been identified to be formed in a time dependent manner within 10 minutes post sampling, such as within 1 minutes or within 3 minutes post sampling, in a comparable test sample.

The present invention also relates to a method of determining the quality of a biological sample by measuring and detecting the presence of fragments of the protein stathmin, which by the present inventors has been identified as one of the first naturally occurring proteins to be degraded post sampling.

As previously reported, biological materials degrade when removed from their natural environment and when being exposed to external factors such as changes in temperature, humidity, pH as well as other physical influences. The degradation of the biological material, i.e., the biological sample, is mostly due to the presence and activity of proteases in the sample. The importance of early degradation of biological samples, i.e., within the very first minutes after the sample has been taken from its natural in vivo source or from a place where it is kept in an inactivated state, impairing the quality of the sample, has not previously been specifically highlighted.

The present inventors show that degradation starts very early post sampling, providing detectable amounts of fragmented proteins already within the very first minutes after a sample has been obtained from a source where it is kept in an inactivated state, or from its natural in vivo source. The presence of protein fragments in the sample may be seen as an indication of impaired quality. As disclosed by the present invention, one of the first proteins to appear in fragments is the protein stathmin, providing a useful marker for early degradation and quality status of the sample.

The present invention provides means for e.g., pharmaceutical companies or academic research groups to determine the quality of a biological sample before initiating expensive and/or time consuming analyses or tests with a biological sample, which may not be of satisfying quality. An easy first test of the sample according to the invention will provide information of the quality of the sample, allowing or dissuading from additional biological tests using that particular sample.

The importance of early protein degradation in a biological sample is shown in a study performed by the inventors wherein the specificity and sensitivity of nanoLC ESI Q-TOF MS was employed for a peptidomic approach to map the peptide content changes in the striatum and the hypothalamus of mouse brain tissue samples at different time-points post-mortem. The inventors also analyzed the brain samples with a proteomic approach utilizing two-dimensional difference gel electrophoresis (2D DIGE) and matrix-assisted laser desorption ionization (MALDI) MS. Mice were sacrificed and the brain proteases were denaturated and inactivated by focussed microwave irradiation at different time-points. The inventors found that the endogenous neuropeptides and neuromodulators, such as neurotensin, enkephalins, dynorphins, substance P were relatively stable up to 10 min post-mortem.

However, more importantly, in contrast to the biologically active neuropeptides and neuromodulators, the degradation of other proteins started immediately. After approximately one min post-mortem a large number of protein fragments were detected in the peptidomic analysis. Furthermore, it was demonstrated that a snap frozen brain (within seconds post-mortem) without microwave irradiation produced the same number of peptides as from a focused microwaved sacrificed mouse brain. These results show that temporally inactivation, such as by freezing of sample can be used if the denaturation is performed directly from the frozen sample.

Post mortem studies of degradation of proteins have been described in many articles but time after death is often counted in hours. Interestingly, many of the protein degradation products reported herein are similar to those described in other publications, where the time after death is much longer (Lametsch et al. 2002) (Fountoulakis et al. 2001).

Among identified proteins that were fragmented in the studies performed by the inventors were hemoglobin, stathmin, cytochrome C oxidase, NADH dehydrogenase, beta-actin, alpha-synuclein, thymosin beta-4, thymosin beta-10, and dihydropyrimidinase-related protein-2 (Sköld et al. 2002). Any of these protein fragments may be measured and detected in a method according to the invention, to determine the quality of a biological sample. It should however be pointed out that this list is not exclusive, and other proteins may be used in a method according to the invention.

The present inventors have shown that analyzing peptides extracted from microwaved tissue using on-line nanoLC/ESI Q-TOF MS and MSMS is a powerful combination for simultaneous detection and identification of a large number of neuropeptides and their post-translational modifications present in the brain, and thus complements standard proteomic methods. However, it is to be understood that in a method according to the invention, any means for detecting the presence of proteins and protein fragments in a biological sample, may be used.

The present invention relates to a method of determining the quality of a biological sample which has been made biologically inactive, wherein said quality is determined by measuring the degradation of one or more proteins and/or peptides present in said sample by detecting the presence of a fragment of said protein and/or peptide in said sample after said inactivation. In one embodiment, said quality is determined by comparing the amount of fragments of said protein and/or peptide with the amount of intact protein or peptide in said sample.

Furthermore, it is to be understood that the quality of the sample may also be determined by detecting only the presence of intact protein in the sample, as well as only the presence of fragments, depending on which means is being used for determining the quality.

Another object of the present invention relates to a method for determining the quality of a biological sample, wherein said quality is determined by detecting the presence and/or amount of a protein or a peptide, where said a protein or peptide has been identified to be degraded in a time dependent manner within 10 minutes post sampling, such as within 1 minutes or within 3 minutes post sampling, in a comparable test sample.

In one embodiment, the invention relates to a method of determining the quality of a biological sample, wherein said protein or peptide is phosphorylated. In one preferred embodiment, said phosphorylated protein or peptide has been identified to be dephosphorylated in a time dependent manner within 10 minutes post sampling, such as within 1 minutes or within 3 minutes post sampling, in a comparable test sample. In another preferred embodiment said peptide is corticotrophin-like intermediate lobe peptide (CLIP).

In one embodiment, a ratio may be calculated between phosphorylated protein or peptide and dephosphorylate protein or peptide. The ratio may be compared to a standard for different in vivo tissues and/or bodily fluids.

In one embodiment, the invention relates to a method of determining the quality of a biological sample, wherein said sample originates from a tissue and/or a bodily fluid.

For certain types of biological samples where separation procedures are needed to obtain the sample, such as plasma and serum samples, it may be necessary to use degradation products that are formed at longer time intervals post sampling as suitable biological markers for the quality of such samples.

Hence, another object of the present invention relates to a method for determining the quality of a biological sample, wherein said quality is determined by detecting the presence and/or amount of a degradation product of a protein or a peptide, where said degradation product has been identified to be formed in a time dependent manner within 60 minutes post sampling, such as within 10 minutes, 20 minutes, 30 minutes, or 45 minutes post sampling, in a comparable test sample.

In a preferred embodiment, the invention relates to a method of determining the quality of a biological sample, wherein said inactivation is performed by denaturation of proteins. In the present context, said denaturation may be performed by physical influences such as heating, freezing, change of pH, mechanical treatment and/or adding pressure to said sample, but is not limited thereto. In the present context, when a sample is heated, a preferred temperature is a temperature within the range 50-100° C., such as within the ranges of 50-60, 60-70, 70-80 or 90-100° C., such as at 50, 60, 70, 75, 80, 85, 90, 95 or 100° C., but it is not limited thereto. Heating may in accordance with the invention be performed by focused microwave irradiation.

Furthermore, when freezing is used to temporarily make the biological sample inactive, temperatures within the range of −0-(−160)° C., such as between −0-(−50), −50-(−100), or −100-(−160)° C., such as −50, −60, −70, −80, −90, −100, −120, −140 or −160° C., are preferably used, but the temperatures are not limited thereto.

Adding pressure to the sample according to invention refers to a process wherein said biological sample is pressurized in the range of 1-12 kbar, such as between 1-5, 5-10, or 10-12 kbar, such as 1, 3, 5, 7, 9, 11 or 12 kbar, but is not limited thereto. The denaturation is performed because proteins are flexible and compressible and loose their secondary dimension structure. Extremes of pH cause denaturation because sensitive areas of the protein molecule acquire more like charges, causing internal repulsion, or perhaps lose charges which were previously involved in attractive forces holding the protein together (Robert K Scopes et al. 1994). Any means as above described may be used for the inactivation process.

Said inactivation may also be performed by denaturating proteins in said sample by adding chemicals such as organic solvents, reducing agents, detergents, and/or chaotropic agents to said sample. Examples of such organic solvents are methanol, acetonitril, and reducing agents, such as acids or bases. Denaturation occurs when pH in the environment differs from the isoelectric point for the specific protein. Most proteins are denatured at pH values ranging from 1-2, as well as between 10-12 (Scopes 1994).

In another embodiment of the invention, said inactivation is performed by inactivation of enzymes in said sample by adding protease inhibitors. Examples of protease inhibitors are serine proteinase inhibitors, cysteine proteinases, alpha-2 macroglobulin, aspartyl protease inhibitors, cysteine protease inhibitors, metalloprotease inhibitors, alpha1-antitrypsin, alpha1-antichymotrypsin, secretory leukocyte protease inhibitor, C-reactive protein, serum amyloid A protein, elasnin 3, elastinal, aprotinin, leupepsin, antipain, pepstatin, phosphoramidon, trypsin inhibitors from albumin or soy beans, gabaxate mesylate, Amastatin, E-64, Antipain, Elastatinal, APMSF, Leupeptin, Bestatin, Pepstatin, Benzamidine, 1,10-Phenanthroline, Chymostatin, Phosphoramidon, 3,4-dichloroisocoumarin, TLCK, DFP, TPCK. In the context of the present invention, any protease inhibitors may be used being suitable for the specific biological sample used in the method, and is not limited to the examples given herein.

It will be understood by the person skilled in the art, that any method which is capable to cause inactivation of the biological sample according to the present invention may be used.

In another embodiment, the invention relates to a method wherein the detected proteins and/or peptides have a distinctive temporal degradation profile.

In yet another embodiment, the invention relates to a method wherein said biological sample originates from a mammalian. Consequently, in one embodiment, the invention also relates to a sample originating from a human.

In another embodiment, the invention relates to a method of determining the quality of a biological sample, as disclosed herein, wherein said quality is determined by detecting the presence of a fragment of a neuropeptide, in said sample. Examples of peptides that may be detected in a method according to the present invention are thymosin beta-4 or thymosin beta-10. The neuropeptides are however not limited thereto. Furthermore, the invention relates to a method, wherein said quality of a biological sample is determined by detecting the presence of a fragment of an acetylated protein. In higher eukaryotes 80-90% of all proteins synthesized in the cytoplasm are isolated with their N-termini acetylated (Second Edition Proteins, Structures and molecular properties, Thomas E. Creighton). Examples of acetylated proteins that may be detected in a method according to the present invention are the following: stathmin, hemoglobin alpha-chain, alpha-synuclein, 14-3-3 protein zeta/delta, alpha enolase, glyceraldehyde 3-phosphate dehydrogenase, serum albumin precursor, protein kinase C, gamma actin, sorcin, FK506-binding protein beta, globin, alpha-globin, and hemoglobin beta (Gevaert et al. 2003, Skold et al. 2002,).

Stathmin is a phosphoprotein which can be found in all tissues that has a cytoskeleton and has also been found in body fluids. The protein is involved in the regulation of the microtubule (MT) filament system by destabilizing microtubules. It prevents assembly and promotes disassembly of microtubules.

In a preferred embodiment, a method as described is comprised within the scope of the present invention, wherein said quality of a biological sample is determined by measuring the degradation of stathmin present in said sample by detecting the presence of a fragment of the protein stathmin (SEQ ID NO:1, SEQ ID NO:3 or SEQ ID NO:4), such as a fragment comprising 19 amino acids of stathmin (SEQ ID NO:2). The present inventors have shown that stathmin is one of the first proteins to be degraded in a biological sample post sampling, and is therefore considered a suitable protein to measure possible degradation products of in a method according to the invention. In another preferred embodiment, the invention relates to a method for determining the quality of a biological sample wherein said peptide fragment is an N-terminal fragment of the protein stathmin. In yet one preferred embodiment, the invention relates to a method for determining the quality of a biological sample wherein said peptide fragment is the peptide SEQ ID NO:2. In another preferred embodiment, the quality of a biological sample is determined by detecting the presence of a specific fragment of stathmin comprising SEQ ID NO:2, and/or a fragment thereof.

In another preferred embodiment the invention relates to determining the quality of a biological sample by detecting a fragment of stathmin which comprises less than 148 amino acids of SEQ ID NO:1, SEQ ID NO:3 or SEQ ID NO:4, such as, but not limited to, a fragment comprising between 1-20, 20-30, 30-50, 50-60, 60-80, 80-90, 90-100, 100-110, 110-120, 120-130 or 130-148 amino acids, such as 10, 15, 20, 25, 30, 35, 40, 45, 50, 55, 60, 65, 70, 75, 80, 85, 90, 95, 100, 110, 120, 125, 130, 140, or 145 amino acids.

Another object of the present invention relates to a method of determining the quality of a biological sample, wherein said quality is determined by measuring the degradation of the protein stathmin (SEQ ID NO:1) in said sample by detecting the presence of a fragment of stathmin. In another embodiment the present invention relates to a method, wherein said quality is determined by measuring the degradation of the protein stathmin as comprised in SEQ ID NO:3 or SEQ ID NO:4. In one embodiment, the amount of stathmin fragments is compared with the amount of intact stathmin to determine the quality of the sample.

Said measurement may optionally be preceded by an inactivation of said sample, in accordance with the invention. Said quality may also be determined by detecting the presence of a fragment of stathmin comprising SEQ ID NO:2, and/or a fragment thereof. In one embodiment, said sample is originating from a mammalian. In another preferred embodiment, said sample is originating from a human.

Stathmin, an N-terminally acetylated protein of 17 kDa, comprising 148 amino acid residues, may be fragmented from its N-terminal post sampling. In such case, an emerging fragment may be rendered by cleavage at a specific site, 19 residues from the N-terminal giving rise to a fragment with a molecular weight of 2105 Da (SEQ ID NO:2). In an experiment performed by the inventors, the fragment is detected one minute post sampling, and may in one embodiment of the invention serve as a quality indicator of sample handling. In one embodiment, a sample to be analyzed for quality is separated and analyzed on a gel next to a protein ladder ranging e.g. from 2 to 20 kDa. Stathmin and the fragment originating from stathmin by N-terminal cleavage, may be detected using an antibody recognizing said fragment. When essentially no fragments are detected on the gel, the sample is of high quality. If fragments are detected, a ratio may be calculated between fragment of stathmin and intact stathmin. The ratio may in one context be compared to a “temporal degradation profile” produced for different tissues and/or body fluids.

In one embodiment of the invention, a cut-off filter is used to separate intact stathmin from degradation fragments of stathmin. Both fractions are analyzed using antibodies to detect fragments of stathmin and/or intact stathmin. When essentially no fragments are detected in the fraction that has passed the filter, the sample is of high quality. If fragments are detected, a ratio may be calculated between fragments of stathmin and intact stathmin. In one context, said ratio is compared to a “temporal degradation profile” obtained and defined for different tissues and/or bodily fluids.

In yet another embodiment of the invention, an affinity column is used to capture intact stathmin and fragments of stathmin. The captured protein/fragment is eluted from the column. The fraction is separated on a size-exclusion column and is detected by e.g., UV light absorbance. When essentially no fragments are detected, the sample is of high quality. If fragments are detected, a ratio may be calculated between the amount of fragments of stathmin and the amount of intact stathmin. The ratio may be compared to a standard for different tissues and/or bodily fluids.

In another embodiment of the invention, intact stathmin is separated from its degradation fragment by chromatographic methods or filtration. Prior to or after the separation, stathmin and its peptides are specifically targeted using e.g., molecules with affinity to stathmin and its fragment e.g., antibodies. Quantifiable detection of the specific polypeptides comprises using size-exclusion chromatography, ultra violet light, dyes e.g. coomassie blue, silver staining or, fluorescence, spectrophotometry, mass spectrometry or protein determination kits e.g. Lowry reaction, Biuret reaction (Lowry, et al, 1951; Goruall et al, 1949) is done to establish the ratio between the concentrations of the protein fraction and the protein fragment fraction.

Obviously, the methods for detection of stathmin and fragments of stathmin described herein may be used for any protein and/or fragment thereof which is to be detected in accordance with the invention.

Another object of the invention is to provide peptides than can be used as biological markers for the quality of a biological sample, such as, but not limited to the peptides, SEQ ID NO: 2, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85.

When any one of these markers, being a fragment of an intact protein as indicated in Table 2, are used, a ratio may be calculated between the amount of fragment and the amount of the corresponding intact protein. The ratio may be compared to a standard for different tissues and/or bodily fluids.

Another object of the invention is to provide an antibody directed to any of the peptides SEQ ID NO: 2, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, for the determination of the quality of a biological sample according to present invention.

In another embodiment, the detection of any protein and/or a fragment in accordance with the invention is performed using mass spectrometry (MS). In yet another embodiment, the detection is performed by gel electrophoresis alone or in combination with mass spectrometry. In yet another embodiment, the detection is performed by two-dimensional difference gel electrophoresis (2D DIGE) alone or in combination with matrix-assisted laser desorption ionization mass spectrometry (MALDI MS). In yet another embodiment the detection is performed by liquid chromatography (LC) alone or in combination with MS. In another embodiment, the detection is performed by (capillary nanoscale) LC alone or in combination with electrospray ionization quadrupole time-of-flight nanoLC/ESI Q-TOF) MS, or nanoLC MALDI MS. In another embodiment the detection is performed by a surface plasmone resonance (SPR) based assay. In yet another embodiment the detection is performed by a quartz crystal microbalance-dissipation (QCM-D) based assay.

In another preferred embodiment, the detection of any protein and/or a fragment in accordance with the invention is performed using antibodies directed to one or more proteins and/or fragments thereof.

Another object of the present invention is provide a method for determining the quality of a biological sample, wherein said quality is determined by detecting the total amount of degradation products of proteins and peptides in said sample, wherein
said degradation products are peptide fragments with a molecular weight less than 10 kDa,
   and
the quality of said biological sample is determined by comparing the total amount of peptide fragments present in the biological sample with the standard amount of peptide fragments and endogenous peptides present in comparable biological samples of high quality.

In one embodiment of the invention, said degradation products are peptide fragments with a molecular weight less than 5 kDa, or preferable less than 3 kDa.

In one embodiment of the invention, said detection is performed using a specific N-terminal or specific C-terminal reagent.

In another embodiment of the invention, the peptide fragments in the biological sample are separated from the proteins and peptides of said sample before detection by means of size exclusion chromatography or ultrafiltration.

In another embodiment of the invention, the ratio between the amount of peptides fragments and the amount of proteins and peptides is calculated, and where said ratio is compared to standard ratios for comparable biological samples of high quality.

In another embodiment, the presence of any protein and/or fragment in a sample is determined by high molecular proteins being separated from low molecular peptides and protein fragments by chromatographic methods or filtration in a sample. Specific sites of the proteins/peptides, e.g., N-terminal, C-terminal, specific amino acids are labeled using applicable technique with a fluorescent, enzymatic, biotin or radioactive label prior to or after the separation. Quantifiable detection of the polypeptides using dyes e.g., coommasie blue, silver staining or ultra violet light, fluorescence, spectrophotometry, mass spectrometry or protein determination kits e.g. Lowry reaction, Biuret reaction (O. H. Lowry et al, 1951; Goruall A G et al, 1949) is carried out to establish the ratio between the concentrations of the protein fraction and the protein fragment fraction.

In yet another embodiment, a cut off filter is used to separate intact proteins from degraded forms of proteins. Both fractions are analyzed, i.e. the filtrate and the retentate using antibodies to detect fragments and intact proteins. A ratio may be calculated between fragments and intact proteins. The ratio may be compared to custom standards for different tissues and body fluids.

In another embodiment, said sample is separated on a size exclusion column and the fragments/proteins are detected by e.g. UV light absorbance. A ratio may be calculated between fragments and intact proteins. The ratio may be compared to standards for different tissues and body fluids. Samples with similar ratio may be compared to each other even if the sample quality is questionable.

Furthermore, in one embodiment of the invention, the detection step is performed at several time-points after the initial measurement to determine the quality of said sample post sampling. Several measurements may be necessary to determine if the quality of the sample has deteriorated post sampling and after presumed inactivation. Such measurements may be performed at any stage after inactivation, such as, but not limited to, within 0-4 hours after the inactivation. The set up of experimental parameters will be specific for each situation, as is well known to the person skilled in the art, and will therefore not be given herein.

In another embodiment, the invention relates to a method for determining the quality of a biological sample according to the invention, which is preceded by the steps of: homogenizing a sample from a bodily fluid and/or a tissue and extracting one or more peptides and/or proteins from said sample.

In yet another embodiment, the invention relates to a method for determining the quality of a biological sample according to the invention, which is preceded by the steps of: taking a biological sample from a bodily fluid and/or a tissue such as from a mammal, e.g., from a human, homogenizing said sample in a buffer solution and extracting peptides from said sample. Said sample taken from a mammal are taken using means appropriate for the source of the biological sample, such as, but not limited to, a biopsy for muscle tissue, a scalpel for the top layer of a tissue, or a syringe for a bodily fluid.

In the present context, said biological sample may be homogenized by any appropriate means rendering the biological sample homogenous and suitable for subsequent steps of the method, and is therefore not limited to the examples given herein. Examples of cell disrupting methods to release proteins into solution of said sample are hand homogenizer, ultrasonication, French press or cell lysis by osmotic disruption (Scopes, 1994)

Extraction of the peptides and/or proteins in the sample may be performed by any appropriate methods which are well-known to the person skilled in the art, and is not limited to the examples given herein. Examples of extraction methods are given e.g. in Protein Purification (Scopes 1994).

In one embodiment of the invention, said biological sample to be used in a method according to the invention is originating from plasma, serum, urine, cerebrospinal fluid and/or a biopsy. It is to be understood that samples for biological analyses may be obtained from any appropriate source and are not limited to the examples given herein.

In one embodiment, the invention also relates to the use of a biological sample according to the invention in a biological, biochemical and/or chemical analysis.

Another object of the invention relates to an antibody for detecting stathmin (SEQ ID NO:1, SEQ ID NO:3 or SEQ ID NO:4) and/or a fragment thereof. The invention also specifically relates to an antibody for detecting a fragment of stathmin comprising SEQ ID NO:2, and/or a fragment thereof. In another embodiment, the invention relates to an antibody capable of detecting a fragment of stathmin which is less than 19 amino acids in length, such as between 1-5, 5-10, 10-15 or 15-18 amino acids, such as 3, 5, 7, 8, 9, 10, 11, 12, 13, 14, 15, 16, 17 or 18 amino acids. In yet another embodiment, the invention relates to detecting a fragment of stathmin which is more than 19 amino acids. It is to be understood that in a method according to the invention, any fragment of stathmin according to the invention, may be detected to determine the quality of a biological sample. An antibody according to the invention may be prepared in accordance with standard methods within the field, such as described in Handbook of experimental immunology (Handbook of experimental immunology, Vol. 2, Cellular immunology, 4. ed, Oxford: Blackwell, 1986) In another embodiment, the invention relates to the use of an antibody for detecting stathmin and/or a fragment thereof, such as SEQ ID NO:2, as disclosed herein, for determining the quality of a biological sample according to the invention.

An antibody according to the invention may be used in a kit for detecting the presence of a specific peptide and/or protein. Said antibody may be used in immunological assays e.g. Western Blot, enzyme linked immunosorbent assay (ELISA), radioimmunoassay (RIA), affinity columns or immunoprecipitation (Handbook of experimental immunology, Vol. 2, Cellular immunology, 4. ed, Oxford: Blackwell, 1986). Additional examples of immunological assays are surface plasmone resonance (SPR), and quartz crystal microbalance-dissipation (QCM-D),

Furthermore, the invention relates to an antibody for detecting an acetylated protein. Examples of acetylated proteins that may be detected in a method according to the present invention are the following stathmin, hemoglobin alpha chain, alpha synuclein, 14-3-3 protein zeta/delta, alpha enolase, glyceraldehyde 3-phosphate dehydrogenase, serum albumin precursor, protein kinase C, gamma actin, sorcin, FK506-binding protein, beta globin, alpha globin, and hemoglobin beta (Gevaert et al. 2003, Skold et al, 2002). In another embodiment the invention relates to the use of an antibody for detecting an acetylated protein as disclosed herein, for determining the quality of a biological sample according to the invention.

In another preferred embodiment, the invention relates to a kit comprising an antibody as described by the invention optionally in combination with suitable reagents, for detecting the presence of protein and/or a fragment thereof in said sample, for determining the quality of a biological sample in accordance with the invention.

In yet another preferred embodiment, the invention relates to a kit comprising an antibody directed to stathmin and/or fragments thereof as disclosed herein, optionally in combination with suitable reagents. In another embodiment, the invention relates to a kit comprising an antibody directed to an acetylated protein optionally in combination with suitable reagents.

In yet another embodiment, the invention relates to a kit for determining the quality of a biological sample, which kit comprises a site recognition molecule, as described by the invention, in combination with suitable reagents, for detecting the presence of protein and/or a fragment thereof in a biological sample, such as stathmin, and/or a fragment thereof.

Suitable reagents may comprise e.g. a standard reference for comparing the amount of fragments and/or the intact proteins to which the antibody is directed to, as well as means to detect the binding of the antibody.

### EXPERIMENTAL SECTION

### Experiment 1

### Post-Mortem Changes of Proteins in Cortex Utilizing Two-Dimensional Difference Gel Electrophoresis

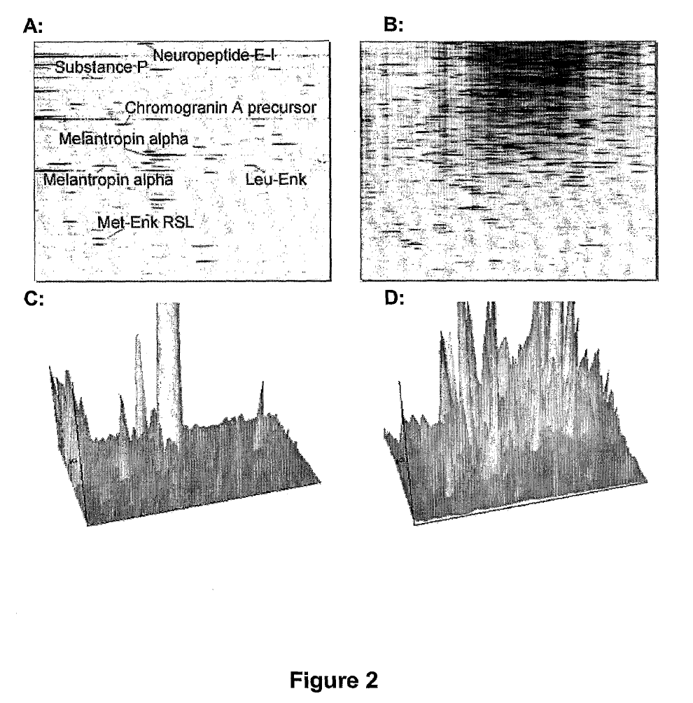

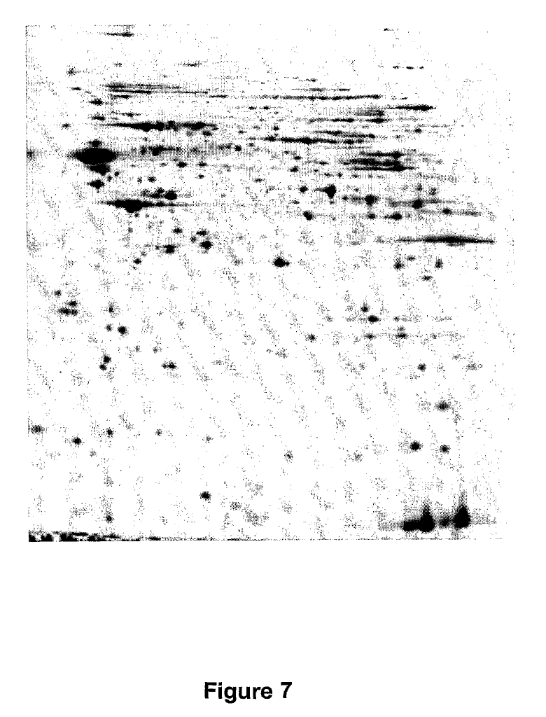

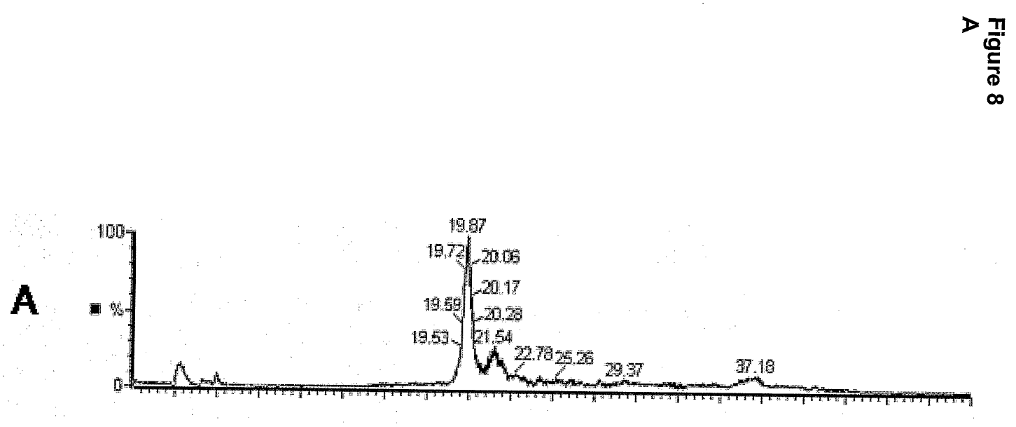

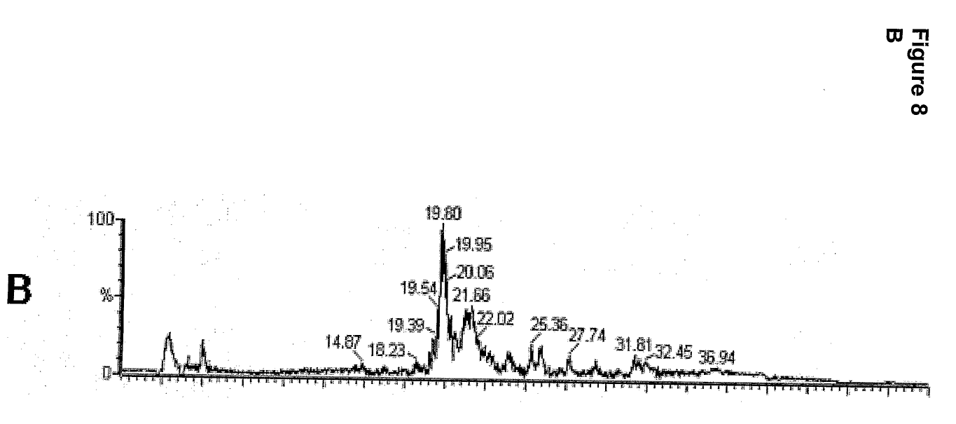

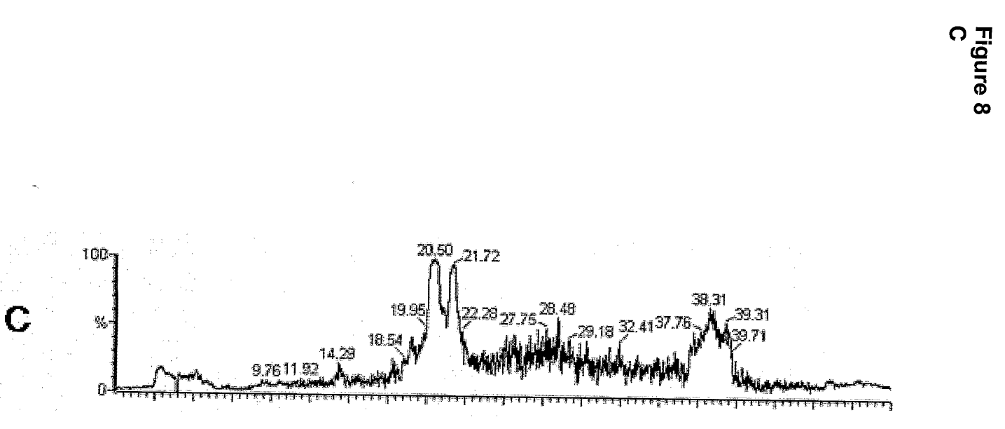

Differences in the levels of proteins due to postmortem degradation processes were studied in mouse brains. A control group were instantly sacrificed by focused microwave irradiation and another group of animals were sacrificed by decapitation and kept at room temperature (22° C.) for 10 min, and was then subjected to focused microwave irradiation. The cortex of the brain was dissected out. The changes in protein levels were studied using two-dimensional difference gel electrophoresis (2D-DIGE) (FIG. 7) and the proteins were identified using nanoLC/ESI LTQ MS (FIG. 2). A number of proteins were found to be significantly changed due to post mortem time (Table 1). The post mortem changes of protein levels using 2D-GE have been studied in a number of publications (Fountoulakis et al (2001) Franzen et al. (2003)) but at much longer post-mortem intervals (hrs).

TABLE 1
Changed proteins

| | |
|---|---|
| Pyruvate dehydrogenase E1 alpha 1 | gi\|6679261\|ref\|NP_032836.1\| |
| Eno1 protein | gi\|34784434\|gb\|AAH56611.1\| |
| Synapsin II | gi\|8567410\|ref\|NP_038709.1\| |
| ATP synthase, H+ transporting | gi\|6680748\|ref\|NP_031531.1\| |
| 3-oxoacid CoA transferase | gi\|18266680\|ref\|NP_077150.1\| |
| ATP synthase, H+ transporting | gi\|6680748\|ref\|NP_031531.1\| |
| Dihydropyrimidinase-like 1 | gi\|3122030\|sp\|P97427\|DPY1_MOUSE |
| Dihydropyrimidinase-like 2 | gi\|6753676\|ref\|NP_034085.1\| |
| Dihydropyrimidinase-like 3 | gi\|6681219\|ref\|NP_033494.1\| |
| T complex polypeptide 1 | gi\|201725\|gb\|AAA40338.1\| |
| Ina protein | gi\|17390900\|gb\|AAH18383.1\| |
| Eukaryotic translation elongation factor 2 | gi\|33859482\|ref\|NP_031933.1\| |
| Elongation factor 2 | gi\|3642667\|gb\|AAC36523.1\| |
| Dynamin-1 | gi\|32172431\|sp\|P39053\|DYN1_MOUSE |
| Dynamin | gi\|487851\|gb\|AAA37318.1\| |
| Serum albumin | gi\|3647327\|emb\|CAA09617.1\| |
| dnaK-type molecular chaperone hsc70 | gi\|476850\|pir∥A45935 |
| Heat shock protein 2 | gi\|31560686\|ref\|NP_032327.2\| |
| DnaK-type molecular chaperone Hsc70t | gi\|2119722\|pir∥I49761 |
| COP9 signalosome subunit 4 | gi\|6753490\|ref\|NP_036131.1\| |
| Creatine kinase, brain | gi\|10946574\|ref\|NP_067248.1\| |
| Tropomodulin 2 | gi\|6934242\|gb\|AAF31669.1\| |
| Gamma-actin | gi\|809561\|emb\|CAA31455.1\| |
| Pyridoxal (pyridoxine, vitamin B6) kinase | gi\|26006861\|ref\|NP_742146.1\| |
| Protein phosphatase 1 | gi\|28173568\|ref\|NP_766295.1\| |

### Experiment 2

### Differential Display of Endogenous Peptides in Striatum

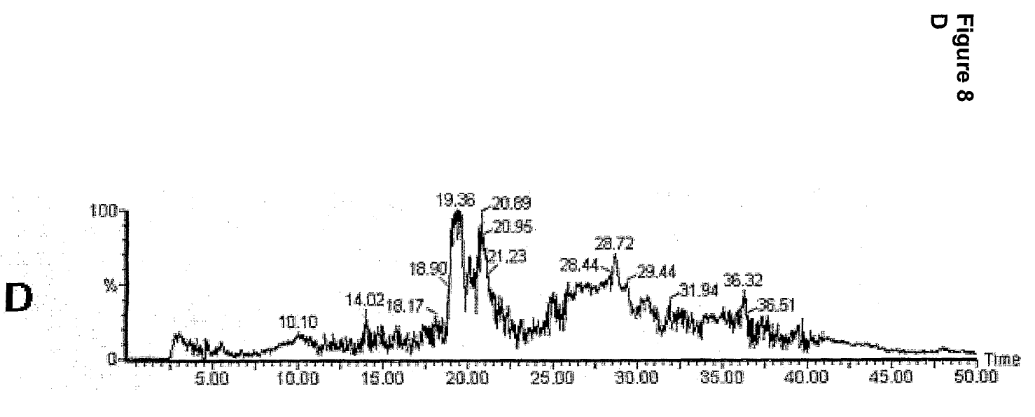

Differences in the levels and numbers of protein fragments and peptides due to post mortem degradation processes were studied in mouse brains. Mice were sacrificed by focused microwave irradiation and another three groups were sacrificed by decapitation kept at room temperatures (22° C.) and was then subjected focused microwave irradiation after 1, 3 and 10 min. The brain area striatum was dissected out. For the analysis of neuropeptides and small proteins <10,000 Da, we utilized nanoLC/ESI Q-TOF MS (FIG. 8). Using this method approximately 550 distinct MS peaks from the instantly deactivated tissue were detected in a single analysis consisting of known neuropeptides, hormones and potential new biological active peptides (Svensson et al.).

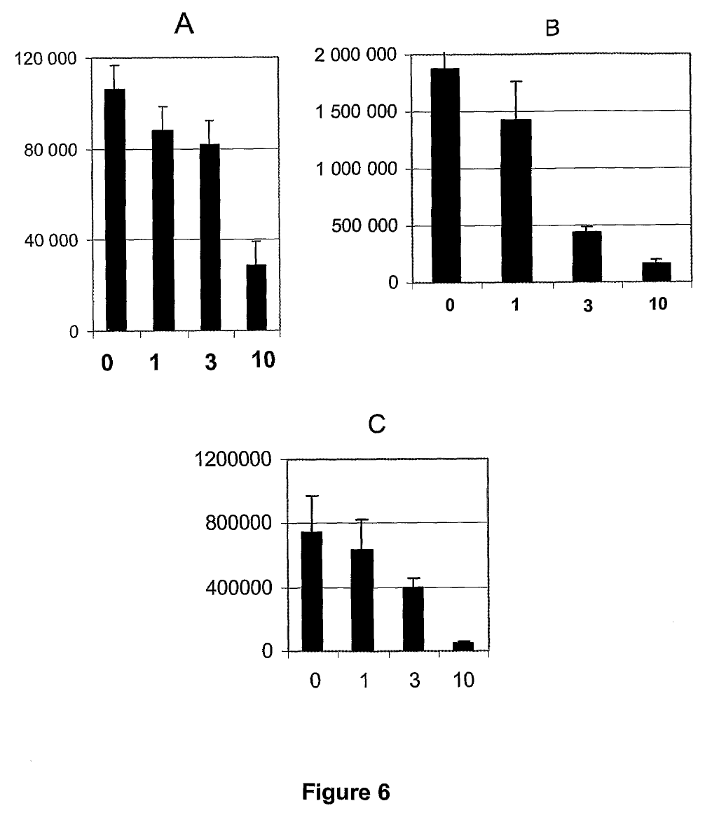

Neuropeptide levels were compared at the different times post mortem. In this study generally, peptides including met-enkephalin, leu-enkephalin, met-enkephalin-RSL, neuropeptide El, and beta-endorphin were presented in higher levels at time point zero and then decreased after the post-mortem time points, 1, 3 and 10 min (FIG. 6). Some peptides increased with post mortem time, including substance P, thymosin beta-10, and novel peptides originated from the peptide precursors VGF and POMC. Some peptides thymosin beta-4, little SAAS, and neurotensin were more stable and did not change over the different post mortem times.

After only one minute post mortem the first protein fragments were detected due to degradation of proteins. At 10 min post-mortem, the protein degradation fragments were the dominating content of the sample. This experiment showed that tissue that has not been proteolytically deactivated, or immediately frozen, is not adequate for protein and peptide analysis (Skold et al., 2002).

Table 2 lists a number of protein fragments that were found to be formed in a time dependent manner within 10 minutes post sampling, i.e. these protein fragments were detected in increasing amounts at 1, 3 and 10 minutes post sampling, but could not be detected at 0 min post sampling.

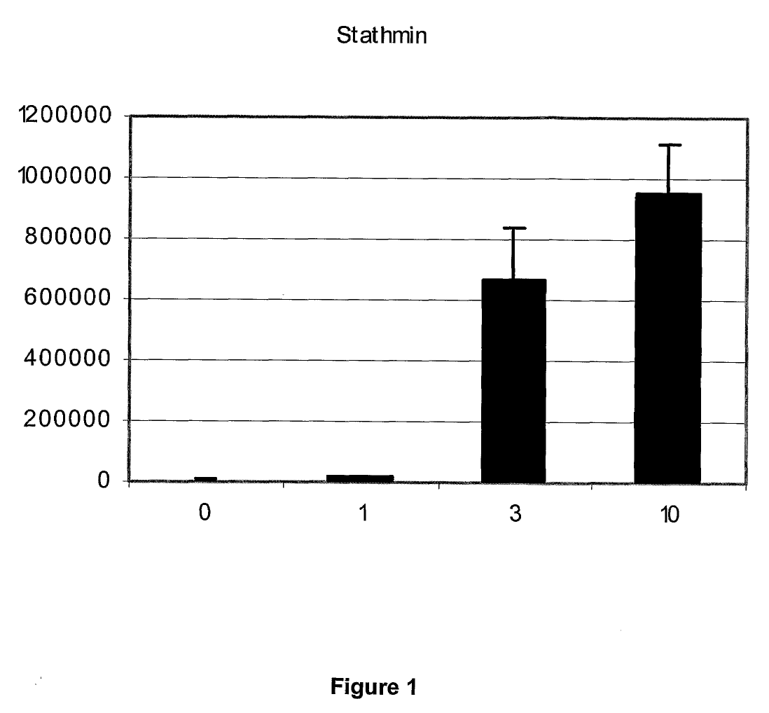

The level of the acetylated fragment from stathmin, SEQ ID NO:2 was found to increase after longer post mortem times and would therefore serve as a excellent marker for protein degradation and post mortem times (FIG. 1). Further, tissue that has been frozen and thawed prior to analysis generally accelerates the degradation process. Therefore, the tissue has to be deactivated proteolytically before freezing or rapidly deactivated proteolytically in its frozen state to enable the relatively low-abundant neuropeptides to remain intact. These procedures minimize degradation of proteins by proteolysis and also conserves the post-translational modifications of the neuropeptides. Previous studies focused on specific peptides have shown that several peptides are present in higher levels after microwave irradiation than after decapitation (Mathe et al., 1990; Nylander et al., 1997; Theodorsson et al., 1990).

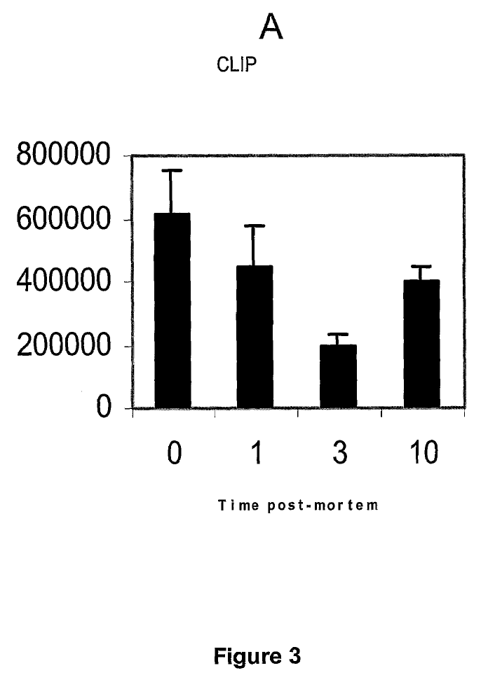

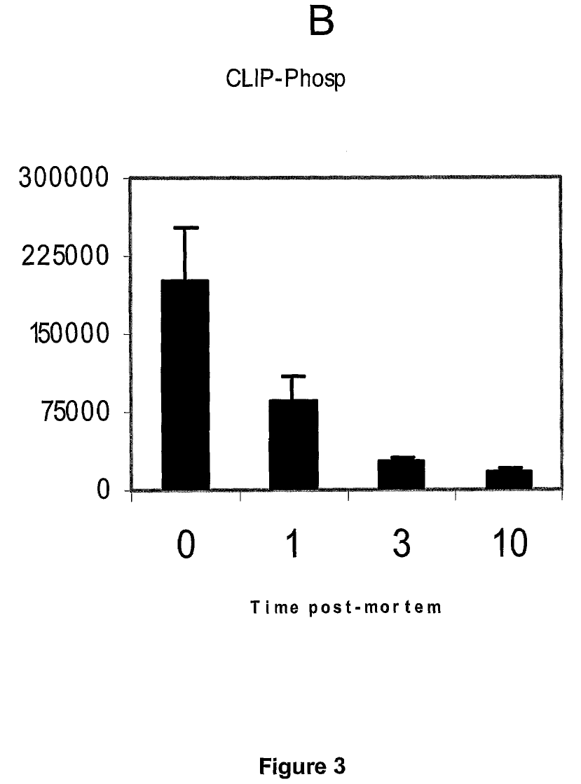

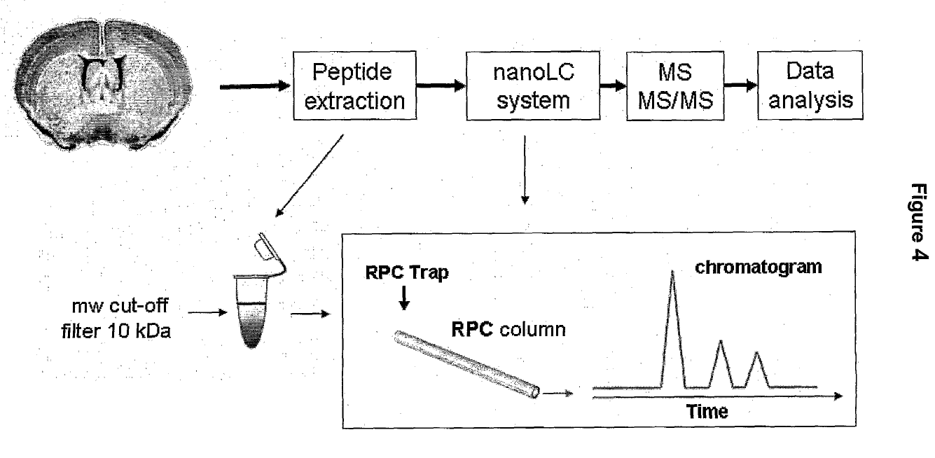

Dephosphorylation of proteins and peptides is a rapid process. Microwave irradiation has been used to prevent dephosphorylation post mortem (Hossain et al, 1994, Li et al, 2003). The neuropeptide corticotrophin-like intermediate lobe peptide (CLIP) was sequenced and identified with and without a phosphate group at Ser154. The levels of phosphorylated CLIP decreased with time post-mortem, whereas the unphosphorylated form of CLIP was relatively stable (FIG. 3). Overall the level of detected neuropeptides in hypothalamus is considerably higher than previously reported.

### Experiment 3

### Blood, Plasma and Liver Peptide Analysis of Stathmin Fragments

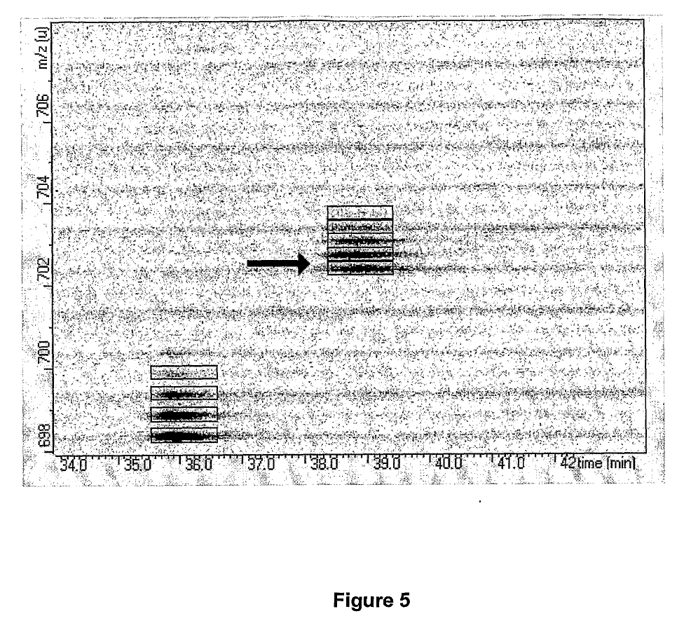

We utilized nanoLC/ESI Q-TOF MS to investigate whether fragments of stathmin and other protein fragments would be present in plasma and/or blood, which had not been rapidly proteolytically deactivated post mortem. The 19 residue peptide fragment from the N-terminal end of stathmin with a molecular weight of 2105 Da (SEQ ID NO:2) were found both in blood and plasma. This indicated that the stathmin fragment could be used for detecting the quality of the sample studied. FIG. 5 shows the fragment of stathmin detected in plasma.

### Materials and Methods

### Sample Preparation

Microwave irradiation was performed in a small animal microwave (Murimachi Kikai, Tokyo, Japan) for 1.4 s at 4.5-5 kW. Mice were sacrificed by cervical dislocation and microwave irradiated or sacrificed by the microwave irradiation directly. Additional animals were also sacrificed by cervical dislocation and the head of the animals were rapidly cooled in liquid nitrogen. Brain areas was dissected out and stored at −80° C. Liver was dissected out and blood was collected directly after cervical dislocation.

For the peptide studies the liver and brain was suspended in cold extraction solution (0.25% acetic acid) and homogenised by microtip sonication (Vibra cell 750, Sonics & Materials Inc., Newtown, Conn.) to a concentration of 0.2 mg tissue/μL. The suspension was centrifuged at 20,000 g for 30 min at 4° C. Blood was prepared by centrifugating down the cells to get the plasma in the same way as for the liver and the brain tissue. The protein- and peptide-containing supernatants was transferred to a centrifugal filter device (Microcon YM-10, Millipore, Bedford, Mass.) with a nominal molecular weight limit of 10,000 Da, and centrifuged at 14,000 g for 45 min at 4° C. Finally, the peptide filtrates was frozen and stored at −80° C. until analysis.

For the protein studies the brain tissue were lysed by sonication in ice-cold lysis buffer (7 M urea, 2 M thiourea, 4% CHAPS, 30 mM TrisCl) at pH 8.5 and centrifuged at 14.000 g at 4° C. for 30 min. The protein concentration of each homogenate was established using Protein Determination Reagent PlusOne 2-D Quant Kit (Amersham Biosciences).

### Nano LC/ESI Q-TOF MS

Five μL peptide filtrate (equivalent to 1.0 mg brain tissue) was injected onto a fused silica capillary column (75 μm i.d., 15 cm length), packed with 3 μm diameter reversed phase C18 particles (NAN75-15-03-C18PM, LC Packings, Amsterdam, the Netherlands). The particle bound sample was desalted by an isocratic flow of buffer A (0.25% acetic acid in water) for 35 min and eluted during a 60 min gradient from buffer A to B (35% acetonitrile in 0.25% acetic acid), delivered using an Ultimate LC system (LC Packings, Amsterdam, the Netherlands). The eluate was directly infused into the ESI Q-TOF mass spectrometer (Q-T of, Micromass Ltd., Manchester, United Kingdom) at a flow rate of 120 mL/min for analysis (Skold et al, 2002).

Data acquisition from the ESI Q-TOF instrument was performed in continuous mode and mass spectra were collected at a frequency of 3.6 GHz and integrated into a single spectrum each second. The time between each such spectrum was 0.1 s. The parameter settings were as follows: Cone 39 V, extractor 3 V, RF lens 1.49, source temperature 80° C. focus 0 V, ion energy 1.8 eV, collision energy 10 eV and multiple channel plate detector (MCP) 2100 V. The cone gas flow rate was set to about 100 L/h. In the wide bandpass quadrupole mode of the mass spectrometer, mass spectra were collected in the mass-to-charge (m/z)-ratio range of 300-1000 Da with a mass resolution of 6400 (FWHM) at m/z 558.31 Da.

### Two-Dimensional Fluorescence Difference Gel Electrophoresis

Lyophilized cyanine dyes (CyDye DIGE Cy2, Cy3, Cy5 (minimal dyes), Amersham Biosciences, Uppsala, Sweden) were reconstituted in dimethylformamide (DMF, Aldrich, Germany) to a concentration of 400 pm/μl. For each homogenate, 50 μg of protein was labeled with 400 pmol of either Cy3 or Cy5. Cy2 was used to label the internal standard, which was prepared from pooled aliquots of equal amounts of the samples. The pooled standard was labeled in bulk in sufficient quantity to include a standard on every gel. A total of 4 gels were run in a set to obtain statistical analysis of the protein expression variation between control and 10 min post mortem.

Prior to isoelectric focusing (IEF) the labeled samples were mixed and added to an equal volume of 2× sample buffer consisting of 7 M urea, 2 M thiourea, 4% CHAPS, 20 mg/ml dithiothreitol, 4% Pharmalyte 3-10 (Amersham Biosciences, Uppsala, Sweden).

### Analytical Gels

All 2-D separations were performed using standard Amersham Biosciences 2-D PAGE apparatus and reagents. In brief, Immobiline DryStrips pH 3-10 nonlinear ×24 cm were used for the first dimension separation with the anodic cup-loading technique. Focussing was carried out using Ettan IPGphor IEF System for a total of 48 KVh. Following IEF, strips were equilibrated with reducing buffer A containing 6 M urea, 1% w/v SDS, 30% v/v glycerol, 100 mM Tris-HCl pH 6.8, with 30 mM DTT for 10 min and subsequently equilibrated with alkylating buffer B containing 6 M urea, 1% w/v SDS, 30% v/v glycerol, 100 mM Tris-HCl pH 6.8, 240 mM iodocetamide for a further 10 min. Second-dimension polyacrylamide gel electrophoresis (SDS-PAGE) was performed using 1.0 mm thick, 12.5% SDS polyacrylamide gels cast for the Ettan DALT system between low-fluorescence glass plates using an Ettan DALT Twelve Separation unit in modified Laemmli buffer (0.2% SDS), at 2 W per gel with constant voltage for 16 h.

### Preparative Gels

To allow mass spectrometric protein identifications, 500 μg of unlabelled pooled standard was loaded using the in-gel rehydration technique and separated as described previously for analytical gels. Prior to gel casting, two fluorescent reference markers were attached to a bind-silane treated glass plate and polymerized with the gel for spot picking. The gel was stained using SYPRO Ruby Protein Gel Stain (Molecular Probes, Eugene, Oreg., USA) according to the manufacturer's instructions. Excess stain was removed by four washes in distilled water over the course of 2 h.

### 2-D DIGE Imaging and Analysis.

The Ettan DIGE gel images and the preparative gel image were scanned (Typhoon 9410, Amersham Biosciences, Uppsala, Sweden) using the following settings: Cy2 (488 nm excitation laser and 540/40 nm emission filter); Cy3 (532 nm excitation laser 580/30 nm emission filter); and Cy5 (633 nm excitation laser and 670/30 nm emission filter). The preparative gel was scanned with excitation laser at 457 nm with emission filter at 610/30 nm.

The Differential In-gel Analysis (DIA) module of DeCyder analysis software (V 5.02.02 Amersham Biosciences, Uppsala, Sweden) was used for image linking the simultaneously detected internal standard to the differentially labelled sample spots on each gel. The resulting image pairs, consisting of pooled standard and a sample from the same gel, allow direct measurement of volume ratios between the standard and the samples. Matching between gels utilizing the in-gel standard from each image pair was performed in DeCyder BVA (Biological Variation Analysis) module. This enables quantitative comparison and statistical analysis of samples between gels based on the relative change of sample to its in-gel internal standard.

### Automated Spot Picking

The proteins spots that met the defined statistical requirements were filtered out using student's t-test and analysis of variance (ANOVA). A picklist composed of spots that demonstrated a significant change (p<0.05) in abundance was created from which the Ettan Spot Handling Workstation (Amersham Biosciences, Uppsala, Sweden) excised the protein containing plugs from the prep gel, using a 1.4 mm picking head. The plugs were washed in 50 mM ammonium bicarbonate and dried prior to digestion with trypsin in 20 mM ammonium bicarbonate (37° C. for 70 min). The peptide fragments were extracted with 50% (v/v) acetonitrile (ACN) in 0.1% (v/v) trifluoroacetic acid (TFA) for 20 min and then dried. Parts of the digests were mixed with an equal volume of 50% ACN, 0.5% TFA saturated with α-cyano-4-hydroxycinnamic acid and 0.3 μl were dispensed onto MALDI targets and the remaining were dried once more.

### Protein Characterization and Identification Using Nanomate™ LTQ

For sequence information an automated nanoelectrospray system NanoMate™ 100 (Advion) coupled to an LTQ ion trap mass spectrometer (Thermo Electron, San Jose, USA) was applied. The spray voltage was 1.8 kV the capillary temperature was 160 C, and 35 units of collision energy were used to obtain fragment spectra. Four MS/MS spectra of the most intense peaks were obtained following each full-scan mass spectrum. The dynamic exclusion feature was enabled to obtain MS/MS spectra on most of the unique peptides.

### Data Analysis.

The sequences of the uninterpreted ESI-MS and ESI-MS/MS-spectra were identified by correlation of the NCBI-protein sequence database (http://www.ncbi.nim.nih.gov) using the TurboSequest algorithm in the Bioworks 3.1 software package (Thermo Finnigan). The non redundant subdatabase of mus musculus was used and the Sequest parameters were as following: partial oxidation of methionine (+16 Da), and cystein (+57 Da). Peptide mass tolerance of 1.5 Da and fragment ions tolerance of 0.35 Da. Trypsin was specified as used enzyme. The identified peptides were further evaluated using charge state versus cross-correlation number (Xcorr). The criteria for positive identification of peptides were Xcorr>1.5 for singly charged ions, Xcorr>2.0 for doubly charged ions, and Xcorr>2.5 for triply charged ions.

### Peptide Characterization and Identification Using Quadrupole Time of Flight.

Sequence information of the peptides was obtained from precursor ions (peptides) by an automatic switching function of the Q-TOF software from MS to MSMS mode. The precursor ions were automatically selected for fragmentation during four nanoLC separations and subsequently put in an exclusion list for 200 s. The switching was intensity dependent with the threshold value set to 12 ion counts. The collision chamber was filled with argon with the inlet pressure set to about 15 psi. The collision energy was ramped from 23-31 eV in 5 s. The collected collision-induced dissociation fragmentation spectra were integrated into a single spectrum twice every second in the m/z-ratio range of 40-1200 Da. These spectra were deconvoluted using MaxEnt3 (MassLynx 3.4, Micromass Ltd.) and interpreted by the BioLynx (MassLynx 3.4) software tools and/or manually. The proposed peptide sequences were compared with the non-redundant database of National Center for Biotechnology Information (NCBI) to establish the peptide identities using Basic Local Alignment Search Tool (BLAST) ‘search for short nearly exact matches’ (http://www.ncbi.nim.nih.gov/BLAST).

### Protein Characterization and Identification by the LTQ.

The sequences of the uninterpreted ESI-MS and ESI-MS/MS-spectra were identified by correlation of the NCBI-protein sequence database (http://www.ncbi.nim.nih.gov) using the TurboSequest algorithm in the Bioworks 3.1 software package (Thermo Finnigan). The non redundant subdatabase of mus musculus was used and the Sequest parameters were as following: partial oxidation of methionine (+16 Da), and cystein (+57 Da). Peptide mass tolerance of 1.5 Da and fragment ions tolerance of 0.35 Da. Trypsin was specified as used enzyme. The identified peptides were further evaluated using charge state versus cross-correlation number (Xcorr). The criteria for positive identification of peptides were Xcorr>1.8 for singly charged ions, Xcorr>2.5 for doubly charged ions, and Xcorr>3.5 for triply charged ions.

TABLE 2

Protein fragments identified from mouse sfriatum extract.

| | | | |
|---|---|---|---|
| Swiss-Prot # | Protein^{a} | Sequence | SEQ ID NO. |

| | | | |
|---|---|---|---|
| P60710/P63260 | Actin, cytoplasmic 1,2 | LVVDNGSGMCK | 5 |
| | | MATAASSSSLEKS | 6 |
| | | IGGSILASLSTFQQ | 7 |
| | | ISKQEYDESGPSIVHRK | 8 |
| | | WISKQEYDESGPSIVHRK | 9 |
| Q8K021 | Secretory carrier-associated membrane | ATGVMSNKTVQTAAANAASTAATSAAQNAFKGNQM | 10 |
| | protein 1 | | |
| Q9D164 | FXYD domain-containing ion transport | ITTNAAEPQK | 11 |
| | regulator 6 precursor | ITTNAAEPQKA | 12 |
| | | ITTNAAEPQKAE | 13 |
| | | ITTNAAEPQKAEN | 14 |
| P99029 | Pemxiredoxin 5, mitochondrial precursor | APIKVGDAIPSVEVF | 15 |
| P01942 | Hemoglobin alpha subunit | LASVSTVLTSKYR | 16 |
| | | FASFPTTKTYFPHF | 17 |
| | | ASHHPADFTPAVHASLDK | 18 |
| | | LASHHPADFTPAVHASLDK | 19 |
| | | LVTLASHHPADFTPAVHAS | 20 |
| | | LVTLASHHPADFTPAVHASLDK | 21 |
| | | LVTLASHHPADFTPAVHASLDKFLASVST | 22 |
| | | LASHHPADFTPAVHASLDKFLAS | 23 |
| | | VTLASHHPADFTPAVHASLDKFLAS | 24 |
| | | VLSGEDKSNIKAAWGKIGGHGAEYGAEALER | 25 |
| | | VLSGEDKSNIKAAWGKIGGHGAEYGAEALERM | 26 |
| P02088/P02089 | Hemoglobin beta-1,2 subunit | LVVYPWTQRY | 27 |
| | | LVVYPWTQRYF | 28 |
| Q00623 | Apolipoprotein A-I precursor | VDAVKDSGRDYVSQFESSSLGQQLN | 29 |
| P10637 | Microtubule-associated protein tau | MVDSPQLATLADEVSASLAKQ | 30 |
| | | VDSPQLATLADEVSASLAKQ | 31 |
| P20357 | Microtubule-associated protein 2 | LESPQLATLAEDVTAALAKQ | 32 |
| | | LESPQLATLAEDVTAALAKQG | 33 |
| P68369/Q922F4 | Tubulin alpha-1,6 chain | HSFGGGTGSGFTS | 34 |
| | | FSETGAGKHVPRA | 35 |
| | | LVFHSFGGGTGSGFTS | 36 |
| | | THSLGGGTGSGMGTLLI | 37 |
| | | SVMPSPKVSDTVVEPYNA | 38 |
| Q9D6F9 | Tubulin beta-4 chain | SGPFGQIFRPDNF | 39 |
| Q62361 | Thyroliberin precursor | LLEAAQEEGAVTPDLPGLEKVQVRPE | 40 |
| Q9Z0P4 | Paralemmin | MGYQNVEDEAETKKVLGLQDTIKA | 41 |
| | | MGYQNVEDEAETKKVLGLQDT | 42 |
| P05201 | Aspartate aminotransferase, cytoplasmic | APPSVFAQVPQAPPVLVFK | 43 |
| | | APPSVFAQVPQAPPVLVFKL | 44 |
| Q60771 | Claudin-11 | YYSSGSSSPTHAKSAHV | 45 |
| Q68ED7 | Mucoepidermoid carcinoma translocated | STEARRQQAQQVSPTLSPLSPITQAV | 46 |
| | protein 1 homolog | | |
| P80313 | T-complex protein 1, eta subunit | VWEPAMVRINALTAASEAA | 47 |
| Q99KB8 | Hydroxyacylglutathione hydrolase | MKVELLPAL | 48 |
| P70699 | Lysosomal alpha-glucosidase precursor | PVLEPGKTEVTGYFPKG | 49 |
| P17751 | Triosephosphate isomerase | NAANVPAGTEWCAPPTAYID | 50 |
| | | NAANVPAGTEWCAPPTAYIDFARQK | 51 |
| P32037 | Solute carrier family 2, facilitated | NSMQPVKETPGNA | 52 |
| | glucose transporter member 3 | | |
| P09411 | Phosphoglycerate kinase 1 | LEGKVLPGVDA | 53 |
| | | LKDCVGPEVENACANPAAGTVI | 54 |
| | | LKDCVGPEVENACANPAAGTVIL | 55 |
| | | FLKDCVGPEVENACANPAAGTVI | 56 |
| | | FLKDCVGPEVENACANPAAGTVIL | 57 |
| P16858 | Glyceraldehyde-3-phosphate dehydrogenase | ISWYDNEYGYSNR | 58 |
| P97450 | ATP synthase coupling factor 6, | KFDDPKFEVIDKPQS | 59 |
| | mitochondrial precursor | | |
| Q9DB20 | ATP synthase O subunit, mitochondrial | FAKLVRPPVQVYGIEGRYAT | 60 |
| | precursor | | |
| Q9D3D9 | ATP synthase delta chain, mitochondrial | FDSANVKQVDVPTLTGAFG | 61 |
| | precursor | AEAAAAPAPAAGPGQMSFTFASPTQ | 62 |
| Q61548 | Clathrin coat assembly protein AP180 | DSAPEVAMPKPDAAPS | 63 |
| | | AFAAPSPASTASPAKAESSGVIDL | 64 |
| P17742 | Peptidyl-prolyl cis-trans isomerase A | ADDEPLGRVSF | 65 |
| | | FDITADDEPLGR | 66 |
| | | DITADDEPLGRVSF | 67 |
| | | DITADDEPLGRVS | 68 |
| | | ANAGPNTNGSQFFICT | 69 |
| | | ANAGPNTNGSQFFICTA | 70 |
| Q9R0P9 | Ubiquitin carboxyl-terminal hydrolase | LLLFPLTAQHENF | 71 |
| | isozyme L1 | | |
| Q9DCT8 | Cysteine-rich protein 2 | IYDKDPEGTVQP | 72 |
| O08553 | Dihydropyrimidinase-related protein 2 | FVTSPPLSPDPTTPDFLNS | 73 |
| Q99LX0 | Protein DJ-1 | AIVEALVGKDMANQVKAPLVLKD | 74 |
| Q66JT5 | Protein C18orf10 homolog | KVFKSKNKILI | 75 |
| P56564 | Excitatory amino acid transporter 1 | IAQDNEPEKPVADSETKM | 76 |
| Q9QZM0 | Ubiguilin-2 | ATEAPGLIPSFAPGVGMGVLGT | 77 |
| P70296 | Phosphatidylethanolamine-binding protein | AGVTVDELGKVLTPTQV | 78 |
| | | AGPLCLQEVDEPPQHAL | 79 |
| P34884 | Macrophage migration inhibitory factor | DMNAANVGWNGSTFA | 80 |
| O70251 | Elongation factor 1-beta | GFGDLKTPAGLQVL | 81 |
| P35455 | Vasopressin-neurophysin 2-copeptin | LAGTRESVDSAKPR | 82 |
| | precursor | VQLAGTRESVDSAKPR | 83 |
| | | VQLAGTRESVDSAKPRVY | 84 |
| | | LVQLAGTRESVDSAKPRVY | 85 |

### REFERENCES

1. Ivanov, V. T., A. A. Karelin, M. M. Philippova, I. V. Nazimov, and V. Z. Pletnev, 1997, Hemoglobin as a source of endogenous bioactive peptides: the concept of tissue-specific peptide pool. Biopolymers, v. 43, p. 171-88.
2. Lametsch, R., P. Roepstorff, and E. Bendixen, 2002, Identification of protein degradation during post-mortem storage of pig meat. Journal of Agricultural and Food Chemistry, v. 50, p. 5508-12.
3. Mathe, A. A., C. Stenfors, E. Brodin, and E. Theodorsson, 1990, Neuropeptides in brain: effects of microwave irradiation and decapitation. Life Sciences, v. 46, p. 287-93.
4. Nylander, I., C. Stenfors, K. Tan_No, A. A. Mathe, and L. Terenius, 1997, A comparison between microwave irradiation and decapitation: basal levels of dynorphin and enkephalin and the effect of chronic morphine treatment on dynorphin peptides. Neuropeptides, v. 31, p. 357-65.
5. Prabakaran, S., J. E. Swatton, M. M. Ryan, S. J. Huffaker, J. T. Huang, J. L. Griffin, M. Wayland, T. Freeman, F. Dudbridge, K. S. Lilley, N. A. Karp, S. Hester, D. Tkachev, M. L. Mimmack, R. H. Yolken, M. J. Webster, E. F. Torrey, and S. Bahn, 2004, Mitochondrial dysfunction in schizophrenia: evidence for compromised brain metabolism and oxidative stress. Mol Psychiatry, v. 9, p. 684-97, 643.
6. Sköld, K., M. Svensson, A. Kaplan, L. Björkesten, J. Åström, and P. E. Andrén, 2002, A neuroproteomic approach to targeting neuropeptides in the brain. Proteomics, v. 2, p. 447-454.
7. Svensson, M., K. Skold, P. Svenningsson, and P. E. Andren, 2003, Peptidomics-based discovery of novel neuropeptides. Journal of Proteome Research, v. 2, p. 213-9.
8. Theodorsson, E., C. Stenfors, and A. A. Mathe, 1990, Microwave irradiation increases recovery of neuropeptides from brain tissues. Peptides, v. 11, p. 1191-7.
9. Thorsell, A., S. H. Gruber, A. A. Mathe, and M. Heilig, 1990, Neuropeptide Y (NPY) mRNA in rat brain tissue: effects of decapitation and high-energy microwave irradiation on post mortem stability. Neuropeptides, v. 35, p. 168-73.
10. Scopes Robert K., Protein Purification, Principles and practice, Third Edition, Springer Advanced Texts in chemistry, Series Editor: Charles R. Cantor, 1994.
11. M. Z. Hossain, L. J. Murphy, E. L. Hertzberg och J. I. Nagy, 1994, Journal of Neurochemistry, 62, 2394-2403.
12. Li J. et al, 2003, Post-mortem interval effects on the phosphorylation of signaling proteins, Neuropsychopharmacology, 28, 1017-1025.
13. Khosravi J. Diamandi A., Bodani U., Khaja N., Krishna R., Pitfalls of immunoassay and sample for IGF-I, 2005, Comparison of different assay methodologies using various fresh and stored serum samples. Clinical Biochemistry 38, 659-666,
14. Che, F-Y., Lim J. Pan H. Biswas R and Fricker L., 2005, Quantitative neuropeptidomics of Microwave-irradiated mouse brain and pituitary. Molecular and Cellular proteomics 4:1391-1405.
15. Franzén B. Yang Y. Sunnemark D., Wickman M., Otterwald J., Oppermann M., Sandberg K., 2003. Dihydropyrimidinase related protein-2 as a biomarker for temperature and time dependent post mortem changes in the mouse brain proteome. Proteomics, 3, 1920-1929.
16. Gevaert K, Goethals M, Martens L, Van Damme J. Staes A, Thomas G R, Vandekerckhove J. 2003, Exploring proteomes and analyzing protein processing by mass spectrometric identification of sorted N-terminal peptides, Nature Biotechnology, 21, 566-569.
17. Lowry, O. H., N. J. Rosebrough, A. L. Farr and R. J. Randall, 1951, J. Biol. Chem. 193: 265.
18. Goruall A G, Bardawill C J. 1949, Determination of serum proteins by means of biuret reaction. J Biol Chem 17, 751-66.
19. Sabudeco A. J et al. 2003, Estimation of post mortem interval using the protein marker cardiac Troponin 1. Forensic Science International 134, 11-16.
20. Fountoulakis M. et al. 2001, Postmortem changes in the level of Brain Proteins. Experimental Neurology, 167: 86-94.

1. A method for identifying a biological marker for the quality of a biological sample comprising the steps;
a) detecting the presence and amount of degradation products of proteins and peptides in a test sample at one or more time points within 10 minutes post sampling, and
b) identifying a degradation product which is formed in a time dependent manner within 10 minutes post sampling as a biological marker for the quality of a biological sample.

2. A method according to claim 1, wherein said detection is performed by a method selected from, mass spectrometry, gel electrophoresis alone or in combination with mass spectrometry, two-dimensional difference gel electrophoresis (2D DIGE) alone or in combination with matrix-assisted laser desorption ionization mass spectrometry, liquid chromatography alone or in combination with mass spectrometry, capillary nanoscale liquid chromatography alone or in combination with electrospray ionization (quadrupole) time-of-flight (nanoLC/ESI Q-TOF) MS, surface plasmone resonance (SPR), quartz crystal microbalance-dissipation (QCM-D), and other immunological assays using antibodies directed to one or more of said protein, peptide and/or degradation product.

3. A method according to any of claims 1 to 2, wherein said sample originates from a tissue or a bodily fluid.

4. A method for determining the quality of a biological sample, wherein said quality is determined by detecting the presence and/or amount of a degradation product of a protein or a peptide, wherein said degradation product has been identified to be formed in a time dependent manner within 10 minutes post sampling in a comparable test sample.

5. A method according to claim 4, wherein said protein or peptide has a distinctive temporal degradation profile.

6. A method according to any of claims 4 to 5, wherein said peptide is a neuropeptide.

7. A method according to any of claims 4 to 5, wherein said protein is an acetylated protein.

8. A method according to any of claims 4 to 7 wherein said degradation product is peptide fragment.

9. A method according to claim 8 wherein said peptide fragment is a fragment of the protein stathmin.

10. A method according to claim 9 wherein said peptide fragment is an N-terminal fragment of the protein stathmin.

11. A method according to claim 10 wherein said peptide fragment is the peptide SEQ ID NO: 2.

12. A method according to any of claims 9 to 11, wherein the amount of a fragment of stathmin is compared with the amount of intact stathmin in said sample.

13. A method according to claim 12, wherein the fragments of stathmin in the biological sample are separated from intact stathmin in said sample before detection by means of size exclusion chromatography or ultrafiltration

14. A method according to any of claims 4 to 13, wherein said sample is taken from plasma, serum, urine, cerebrospinal fluid and/or a tissue biopsy

15. A method according to any of claims 4 to 14, wherein said biological sample is of mammalian origin.

16. A method according to claim 15, wherein said sample is of human origin.

17. A method according to any of claims 4 to 16, wherein said detection is preceded by an inactivation of said sample.

18. A method according to any of claims 4 to 17, wherein said detection is performed by a method selected from mass spectrometry, gel electrophoresis alone or in combination with mass spectrometry, two-dimensional difference gel electrophoresis (2D DIGE) alone or in combination with matrix-assisted laser desorption ionization mass spectrometry, liquid chromatography alone or in combination with mass spectrometry, capillary nanoscale liquid chromatography alone or in combination with electrospray ionization (quadrupole) time-of-flight (nanoLC/ESI Q-TOF) MS, surface plasmone resonance (SPR), quartz crystal microbalance-dissipation (QCM-D), and other immunological methods using antibodies directed to one or more of said protein, peptide and/or degradation product.

19. The peptide SEQ ID NO: 2.

20. An antibody directed to the peptide SEQ ID NO: 2.

21. Use of an antibody according to claim 20, for determining the quality of a biological sample.

22. A kit for determining the quality of a biological sample, comprising an antibody to stathmin, optionally in combination with suitable reagents, for detecting the presence of stathmin and/or a fragment of stathmin in said sample.

23. A kit according to claim 22, comprising the antibody according to claim 20.

24. A biological marker for determining the quality of a biological sample, characterized by:
a) being formed post sampling as a degradation product of a protein or a peptide present in said biological sample, and
b) being formed in a time dependent manner within 10 minutes post sampling in a comparable untreated test sample at 25° C.

25. A biological marker according to claim 24, wherein said protein or peptide has a distinctive temporal degradation profile.

26. A biological marker according to any of claims 24 to 25, wherein said peptide is a neuropeptide.

27. A biological marker according to any of claims 24 to 25, wherein said protein is an acetylated protein.

28. A biological marker according to any of claims 24 to 27 which is a peptide fragment.

29. A biological marker according to claim 28 which is a fragment of the protein stathmin.

30. A biological marker according to claim 29 which is an N-terminal fragment of the protein stathmin.

31. A biological marker according to claim 30 which is the peptide SEQ ID NO: 2.

32. A method for determining the quality of a biological sample, wherein said quality is determined by detecting the total amount of degradation products of proteins and peptides in said sample, wherein
a) said degradation products are peptide fragments with a molecular weight less than 10 kDa, and
b) the quality of said biological sample is determined by comparing the total amount of peptide fragments present in the biological sample with the standard amount of peptide fragments and endogenous peptides present in comparable biological samples of high quality.

33. A method according to claim 32, wherein the ratio between the amount of peptides fragments and the amount of proteins and peptides is calculated, and wherein said ratio is compared to standard ratios for comparable biological samples of high quality.

34. A method according to any of claims 32 to 33, wherein said degradation products are peptide fragments with a molecular weight less than 5 kDa, preferably less than 3 kDa.

35. A method according to any of claims 32 to 34, wherein said detection is performed using a specific N-terminal or specific C-terminal reagent.

36. A method according to any of claims 32 to 35, wherein the peptide fragments in the biological sample are separated from the proteins and peptides of said sample before detection by means of size exclusion chromatography or ultrafiltration.


SEQUENCE LISTING (85 sequences)

SEQ ID NO: 1 - protein, 148 aa, Homo sapiens
ASSDIQVKELEKRASGQAFELILSPRSKESVPEFPLSPPKKKDLSLEEIQKKLEAAEERRKSHEAEVLKQLAEKREHEKEVLQKAIEENNNFSKMAEEKLTHKMEANKENREAQMAAKLERLREKDKHIEEVRKNKESKDPADETEAD

SEQ ID NO: 2 - protein, 19 aa, Homo sapiens
  MOD_RES (1)..(1): ACETYLATION
ASSDIQVKELEKRASGQAF

SEQ ID NO: 3 - protein, 148 aa, Mus musculus
ASSDIQVKELEKRASGQAFELILSPRSKESVPDFPLSPPKKKDLSLEEIQKKLEAAEERRKSHEAEVLKQLAEKREHEKEVLQKAIEENNNFSKMAEEKLTHKMEANKENREAQMAAKLERLREKDKHVEEVRKNKESKDPADETEAD

SEQ ID NO: 4 - protein, 148 aa, Rattus norvegicus
ASSDIQVKELEKRASGQAFELILSPRSKESVPEFPLSPPKKKDLSLEEIQKKLEAAEERRKSHEAEVLKQLAEKREHEKEVLQKAIEENNNFSKMAEEKLTHKMEANKENREAQMAAKLERLREKDKHVEEVRKNKESKDPADETEAD

SEQ ID NO: 5 - protein, 11 aa, Mus musculus
LVVDNGSGMCK

SEQ ID NO: 6 - protein, 13 aa, Mus musculus
MATAASSSSLEKS

SEQ ID NO: 7 - protein, 14 aa, Mus musculus
IGGSILASLSTFQQ

SEQ ID NO: 8 - protein, 17 aa, Mus musculus
ISKQEYDESGPSIVHRK

SEQ ID NO: 9 - protein, 18 aa, Mus musculus
WISKQEYDESGPSIVHRK

SEQ ID NO: 10 - protein, 35 aa, Mus musculus
ATGVMSNKTVQTAAANAASTAATSAAQNAFKGNQM

SEQ ID NO: 11 - protein, 10 aa, Mus musculus
ITTNAAEPQK

SEQ ID NO: 12 - protein, 11 aa, Mus musculus
ITTNAAEPQKA

SEQ ID NO: 13 - protein, 12 aa, Mus musculus
ITTNAAEPQKAE

SEQ ID NO: 14 - protein, 13 aa, Mus musculus
ITTNAAEPQKAEN

SEQ ID NO: 15 - protein, 15 aa, Mus musculus
APIKVGDAIPSVEVF

SEQ ID NO: 16 - protein, 13 aa, Mus musculus
LASVSTVLTSKYR

SEQ ID NO: 17 - protein, 14 aa, Mus musculus
FASFPTTKTYFPHF

SEQ ID NO: 18 - protein, 18 aa, Mus musculus
ASHHPADFTPAVHASLDK

SEQ ID NO: 19 - protein, 19 aa, Mus musculus
LASHHPADFTPAVHASLDK

SEQ ID NO: 20 - protein, 19 aa, Mus musculus
LVTLASHHPADFTPAVHAS

SEQ ID NO: 21 - protein, 22 aa, Mus musculus
LVTLASHHPADFTPAVHASLDK

SEQ ID NO: 22 - protein, 29 aa, Mus musculus
LVTLASHHPADFTPAVHASLDKFLASVST

SEQ ID NO: 23 - protein, 23 aa, Mus musculus
LASHHPADFTPAVHASLDKFLAS

SEQ ID NO: 24 - protein, 25 aa, Mus musculus
VTLASHHPADFTPAVHASLDKFLAS

SEQ ID NO: 25 - protein, 31 aa, Mus musculus
VLSGEDKSNIKAAWGKIGGHGAEYGAEALER

SEQ ID NO: 26 - protein, 32 aa, Mus musculus
VLSGEDKSNIKAAWGKIGGHGAEYGAEALERM

SEQ ID NO: 27 - protein, 10 aa, Mus musculus
LVVYPWTQRY

SEQ ID NO: 28 - protein, 11 aa, Mus musculus
LVVYPWTQRYF

SEQ ID NO: 29 - protein, 25 aa, Mus musculus
VDAVKDSGRDYVSQFESSSLGQQLN

SEQ ID NO: 30 - protein, 21 aa, Mus musculus
MVDSPQLATLADEVSASLAKQ

SEQ ID NO: 31 - protein, 20 aa, Mus musculus
VDSPQLATLADEVSASLAKQ

SEQ ID NO: 32 - protein, 20 aa, Mus musculus
LESPQLATLAEDVTAALAKQ

SEQ ID NO: 33 - protein, 21 aa, Mus musculus
LESPQLATLAEDVTAALAKQG

SEQ ID NO: 34 - protein, 13 aa, Mus musculus
HSFGGGTGSGFTS

SEQ ID NO: 35 - protein, 13 aa, Mus musculus
FSETGAGKHVPRA

SEQ ID NO: 36 - protein, 16 aa, Mus musculus
LVFHSFGGGTGSGFTS

SEQ ID NO: 37 - protein, 17 aa, Mus musculus
THSLGGGTGSGMGTLLI

SEQ ID NO: 38 - protein, 18 aa, Mus musculus
SVMPSPKVSDTVVEPYNA

SEQ ID NO: 39 - protein, 13 aa, Mus musculus
SGPFGQIFRPDNF

SEQ ID NO: 40 - protein, 26 aa, Mus musculus
LLEAAQEEGAVTPDLPGLEKVQVRPE

SEQ ID NO: 41 - protein, 24 aa, Mus musculus
MGYQNVEDEAETKKVLGLQDTIKA

SEQ ID NO: 42 - protein, 21 aa, Mus musculus
MGYQNVEDEAETKKVLGLQDT

SEQ ID NO: 43 - protein, 19 aa, Mus musculus
APPSVFAQVPQAPPVLVFK

SEQ ID NO: 44 - protein, 20 aa, Mus musculus
APPSVFAQVPQAPPVLVFKL

SEQ ID NO: 45 - protein, 17 aa, Mus musculus
YYSSGSSSPTHAKSAHV

SEQ ID NO: 46 - protein, 26 aa, Mus musculus
STEARRQQAQQVSPTLSPLSPITQAV

SEQ ID NO: 47 - protein, 19 aa, Mus musculus
VWEPAMVRINALTAASEAA

SEQ ID NO: 48 - protein, 9 aa, Mus musculus
MKVELLPAL

SEQ ID NO: 49 - protein, 17 aa, Mus musculus
PVLEPGKTEVTGYFPKG

SEQ ID NO: 50 - protein, 21 aa, Mus musculus
NAANVPAGTEVVCAPPTAYID

SEQ ID NO: 51 - protein, 26 aa, Mus musculus
NAANVPAGTEVVCAPPTAYIDFARQK

SEQ ID NO: 52 - protein, 13 aa, Mus musculus
NSMQPVKETPGNA

SEQ ID NO: 53 - protein, 11 aa, Mus musculus
LEGKVLPGVDA

SEQ ID NO: 54 - protein, 22 aa, Mus musculus
LKDCVGPEVENACANPAAGTVI

SEQ ID NO: 55 - protein, 23 aa, Mus musculus
LKDCVGPEVENACANPAAGTVIL

SEQ ID NO: 56 - protein, 23 aa, Mus musculus
FLKDCVGPEVENACANPAAGTVI

SEQ ID NO: 57 - protein, 24 aa, Mus musculus
FLKDCVGPEVENACANPAAGTVIL

SEQ ID NO: 58 - protein, 13 aa, Mus musculus
ISWYDNEYGYSNR

SEQ ID NO: 59 - protein, 15 aa, Mus musculus
KFDDPKFEVIDKPQS

SEQ ID NO: 60 - protein, 20 aa, Mus musculus
FAKLVRPPVQVYGIEGRYAT

SEQ ID NO: 61 - protein, 19 aa, Mus musculus
FDSANVKQVDVPTLTGAFG

SEQ ID NO: 62 - protein, 25 aa, Mus musculus
AEAAAAPAPAAGPGQMSFTFASPTQ

SEQ ID NO: 63 - protein, 17 aa, Mus musculus
DSAPEVAAAPKPDAAPS

SEQ ID NO: 64 - protein, 24 aa, Mus musculus
AFAAPSPASTASPAKAESSGVIDL

SEQ ID NO: 65 - protein, 11 aa, Mus musculus
ADDEPLGRVSF

SEQ ID NO: 66 - protein, 12 aa, Mus musculus
FDITADDEPLGR

SEQ ID NO: 67 - protein, 14 aa, Mus musculus
DITADDEPLGRVSF

SEQ ID NO: 68 - protein, 13 aa, Mus musculus
DITADDEPLGRVS

SEQ ID NO: 69 - protein, 16 aa, Mus musculus
ANAGPNTNGSQFFICT

SEQ ID NO: 70 - protein, 17 aa, Mus musculus
ANAGPNTNGSQFFICTA

SEQ ID NO: 71 - protein, 13 aa, Mus musculus
LLLFPLTAQHENF

SEQ ID NO: 72 - protein, 12 aa, Mus musculus
IYDKDPEGTVQP

SEQ ID NO: 73 - protein, 19 aa, Mus musculus
FVTSPPLSPDPTTPDFLNS

SEQ ID NO: 74 - protein, 23 aa, Mus musculus
AIVEALVGKDMANQVKAPLVLKD

SEQ ID NO: 75 - protein, 11 aa, Mus musculus
KVFKSKNKILI

SEQ ID NO: 76 - protein, 18 aa, Mus musculus
IAQDNEPEKPVADSETKM

SEQ ID NO: 77 - protein, 22 aa, Mus musculus
ATEAPGLIPSFAPGVGMGVLGT

SEQ ID NO: 78 - protein, 17 aa, Mus musculus
AGVTVDELGKVLTPTQV

SEQ ID NO: 79 - protein, 17 aa, Mus musculus
AGPLCLQEVDEPPQHAL

SEQ ID NO: 80 - protein, 15 aa, Mus musculus
DMNAANVGWNGSTFA

SEQ ID NO: 81 - protein, 14 aa, Mus musculus
GFGDLKTPAGLQVL

SEQ ID NO: 82 - protein, 14 aa, Mus musculus
LAGTRESVDSAKPR

SEQ ID NO: 83 - protein, 16 aa, Mus musculus
VQLAGTRESVDSAKPR

SEQ ID NO: 84 - protein, 18 aa, Mus musculus
VQLAGTRESVDSAKPRVY

SEQ ID NO: 85 - protein, 19 aa, Mus musculus
LVQLAGTRESVDSAKPRVY